(19) United States
(12) Patent Application Publication
(10) Pub. No.: US 2005/0175709 A1
(43) Pub. Date: Aug. 11, 2005

(54) Therapeutic microparticles

(76) Inventors: Ace M. Baty III, Flagstaff, AZ (US); Robert L. Cleek, Flagstaff, AZ (US); Daniel F. Davidson, Flagstaff, AZ (US); Melissa J. Matzen, Flagstaff, AZ (US); Michael J. Vonesh, Flagstaff, AZ (US); Joshua D. Williams, Tucson, AZ (US)

Correspondence Address: GORE ENTERPRISE HOLDINGS, INC., 551 PAPER MILL ROAD, P. O. BOX 9206, NEWARK, DE, 19714-9206, US

(21) Appl. No.: 11/008,820

(22) Filed: Dec. 9, 2004

Related U.S. Application Data
(60) Provisional application No. 60/529,207, filed on Dec. 11, 2003.

(51) Int. Cl. A61K 9/14

(52) U.S. Cl. 424/489

(57) ABSTRACT

Biodegradable, compression resistant microparticles adapted for injection through a catheter system, such as is useful for selective embolization procedures. The microparticles can optimally be neutrally buoyant relative to a target bodily fluid. Various active agents may be included in the microparticles, such an anesthetic which can reduce pain during an embolization procedure. The invention further comprises methods and equipment for testing and delivering compression resistant microparticles.

### CROSS REFERENCE TO RELATED APPLICATIONS

This is a non-provisional application of provisional application Ser. No. 60/529,207 filed Dec. 11, 2003.

### BACKGROUND OF THE INVENTION

1. Field of the Invention

The present invention relates to the field of using injectable particles, and especially microparticles, to treat a variety of illnesses and other medical conditions.

2. Description of Related Art

Embolotherapy is a minimally invasive procedure performed to treat a variety of vascular pathologies, including the preoperative management of hypervascularized tumors, and arteriovenous malformations. Hypervascularized tumors have abnormally large numbers of blood vessels providing circulation and are either malignant or benign. Arteriovenous malformations are abnormal connections between arteries and veins whose presence can lead to stroke and death. Hypervascularized tumors and arteriovenous malformations can occur in the brain, breast, liver, uterus, ovaries, spine, head and neck and other locations of a body. These maladies occur in both humans and animals.

Embolotherapy has historically been employed as a preoperative adjunctive procedure. Intentionally obstructing the vasculature supply of a tumor requiring surgical excision results in reduced blood loss and procedural complications. An intentional obstruction of the vascular supply, for example, can induce localized ischemia of the tumor, arrest tumor growth, and induce volumetric shrinkage of the tumor.

Embolic agents are generally delivered to a designated area of the body through a catheter device.

Clinical experience in embolotherapy reveals that some known embolic agents are not capable of sufficient accuracy of delivery, are not structurally acceptable, exhibit clumping, clog delivery devices, have unacceptable buoyancies, and/or can negatively affect the vasculature of the patient.

Non-resorbable polyvinyl alcohol (PVA) foam particles have been employed as embolic agents. PVA-foam embolic agents can fragment, aggregate, or clump in a blood vessel during use and such malperformance can occur prior to reaching a desired embolization location. This undesirable behavior can cause blockages resulting in unintended occlusion of a vessel and of the delivery device. Even in cases where the PVA-foam embolic agent forms an embolism at a desired location, the irregular size and shape of the PVA foam embolic agents may prevent full occlusion of the embolism, allowing blood flow to circumvent the ineffective PVA-foam embolic material and to continue feeding the tumor. Known methods of embolotherapy can result in improper, incomplete or ineffective occlusions of the blood supply to targeted tumors, as well as undesired necrosis, or death, of the surrounding tissues. Complications can result in ineffective treatment.

### BRIEF SUMMARY OF THE INVENTION

The present invention provides advances in embolic agent technology and embolotherapy. The materials, microparticles, treatments, equipment and procedures disclosed herein can be utilized in male and female humans and in animals.

In one aspect, the present invention includes a catheter deliverable microparticle having at least one bioabsorbable and/or bioresorbable base polymer a void volume, and exhibiting compression resistance. A compression resistant catheter deliverable microparticle can be engineered to be substantially neutrally buoyant relative to a target bodily fluid or injection media. A compression resistant catheter deliverable microparticle can be utilized as an embolic agent. A catheter deliverable compression resistant microsphere can optionally be size-matched to a target vessel, as well as being suitable for lodgment in a target vessel.

Compression resistant catheter deliverable microparticles can have one or more additives, one or more bioactive agents (e.g., therapeutic agents), or combinations thereof. Further, in some embodiments, a compression resistant catheter deliverable microparticle can have at least one coating. Such coatings may also have one or more additives, bioactive agents, or combinations thereof.

In one embodiment, a microparticle adapted for catheter delivery has at least one copolymer of a monomer having at least a trimethylene carbonate moiety. In another embodiment, a microparticle adapted for catheter delivery has a homopolymer or copolymer of at least a poly(a-hydroxy ester) having a trimethylene carbonate moiety. In yet another embodiment, a catheter deliverable microparticle has a void volume and a void distribution, which is compatible with the microparticle being compression resistant. Voids, void volumes, and void distributions can each, or in combination, be manipulated to contribute to the compression resistance of microparticles.

Buoyancy is another factor that can be manipulated in catheter deliverable compression resistant microspheres. In one embodiment, a catheter deliverable compression resistant microparticle has a specific gravity that is neutrally buoyant relative to a target bodily fluid.

Yet another factor of relevance is that the surface topography of the microspheres can be manipulated to foster, among other things, degradation rate and mode as well as bioactive release kinetics.

Some embodiments of the present invention utilize a large collection or “bolus” of microparticles (referred to herein as a “bolus”). One embodiment of such a bolus has a number of catheter deliverable compression resistant microparticles, each having at least one bioresorbable base polymer and a void volume. The void volume can exist anywhere at the surface and/or inside of the microparticle and can comprise one or more individual voids. In another embodiment, the bolus has a therapeutically effective number of microparticles that are neutrally buoyant relative to a target bodily fluid. Microparticles employed in a bolus can optionally include at least one additive, at least one bioactive agent, or a combination thereof. A bolus may also have sufficient amount of drug-delivering microparticles in order to deliver a pharmaceutically effective drug dose to the patient Embodiments of the present invention may also include any combination of the above mentioned features.

In another embodiment of the present invention, the bolus includes microparticles with a density between 0.9 g/cc and 1.4 g/cc. In still another embodiment, the bolus includes microparticles with a specific gravity of 0.6 to 1.4 relative to a target bodily fluid. In yet another embodiment, the bolus includes microparticles with a void volume of 0 vol % to 98 vol %.

With regard to compression resistance, it is deemed desirable to provide microparticles of the present invention that are resistant to compression. This provides, inter alia, thoroughly predictable behavior when the microparticles are injected into the targeted site in a body. In one embodiment of the present invention the bolus includes microparticles having a given microparticle external diameter (“diameter”) and being resistant to a deformation of their respective external diameters by greater than 10%. In some embodiments compression resistance is evident in that a deformation of a microparticle external diameter of more than 5%, 10% or 20% respectively results in fracturing or mechanical damage to the microsphere.

Microparticles of the present invention can be provided in multiple phases. In one embodiment, a catheter deliverable compression resistant microparticle of the present invention has at least one bioresorbable base polymer, a second material which is different from the bioresorbable base polymer, a void volume in which the second material is optionally present, and a specific gravity of 0.6 to 1.4 relative to a target bodily fluid.

The present invention also includes an embolic microsphere delivery system having a bolus of catheter deliverable compression resistant microspheres and a delivery apparatus containing the bolus configured to inject the bolus of microspheres and a carrier solution into a patient.

In another aspect, the present invention includes an apparatus for testing microparticle compression resistance that utilizes a bolus of microparticles suspended in a carrier solution forming an injectate passed through a test channel under pressure. One embodiment of testing for compression resistance utilizes the steps of: (1) injecting a bolus of microparticles suspended in a carrier solution through a test channel of a defined constricted dimension, the channel having a feed end and an effluent end; (2) observing whether the microparticles exiting the effluent end of the test channel are intact; (3) classifying microparticles which exit the effluent end of the test channel intact as “compressible” if larger than the defined constricted dimension; and (4) classifying any microparticles that are not intact or do not pass through the test channel as “compression resistant.”

### BRIEF DESCRIPTION OF THE SEVERAL VIEWS OF THE DRAWINGS

The operation of the present invention should become apparent from the following description when considered in conjunction with the accompanying drawings, in which:

FIG. 1A is a schematic representation of a microparticle of the present invention illustrating a base polymer and void features;

FIG. 1B is a schematic representation of a microparticle of the present invention illustrating a base polymer and voids containing bioactive agent or additive;

FIG. 1C is a schematic representation of a microparticle of the present invention illustrating a base polymer mixed with bioactive agent or additive and having voids containing bioactive agents or additives;

FIG. 1D is a schematic representation of a microparticle of the present invention illustrating a base polymer mixed with bioactive agent or additive and further including voids;

FIG. 1E is a schematic representation of a microparticle of the present invention illustrating a base polymer having a coating of bioactive agent or additive and having voids;

FIG. 1F is a schematic representation of a microparticle of the present invention illustrating a base polymer mixed with bioactive agent or additive and having both a coating and voids;

FIG. 2A is a scanning electron micrograph (“SEM”), imaged at 150× magnification, of a cross-section of one embodiment of microparticles of the present invention, the microparticles being loaded with lidocaine and having a smooth outer surface;

FIG. 2B is an SEM, imaged at 500× magnification, of a cross-section of microparticles of FIG. 2A;

FIG. 3A is an SEM, imaged at 150× magnification, of a cross-section of another embodiment of microparticles of the present invention loaded with lidocaine and having a microporous outer surface;

FIG. 3B is an SEM, imaged at 500× magnification, of a cross-section of microparticles of FIG. 3A;

FIG. 4A is an SEM, imaged at 150× magnification, of a cross-section of an embodiment of microparticles of the present invention loaded with lidocaine and having a “brain-like” convoluted outer surface;

FIG. 4B is an SEM, imaged at 500× magnification, of a cross-section of microparticles of FIG. 4A;

FIG. 5 is a schematic representation of one embodiment of an in vitro mechanism for testing of compression resistance of the microparticles of the present invention;

FIG. 6A is a schematic representation of a conventional compressible microparticle as it might appear in a small blood vessel;

FIG. 6B is a schematic representation of an embodiment of a compression resistant microparticle of the present invention as it might appear in a small blood vessel, with the blood vessel undergoing some deformation to accommodate the non-compressible microparticle;

FIG. 7 is an enlarged light micrograph, at about 25× magnification, of a longitudinal cross-section of an arterial segment embolized with microparticles of the present invention;

FIG. 8 is an enlarged light micrograph, at about 10× magnification, of a longitudinal cross-section of microparticles of the present invention lodged in vascular structures of a canine kidney;

FIG. 9A is a light micrograph of a microparticle of the present invention showing an absence of internal voids, the particles being approximately 100 to 150 microns in diameter;

FIG. 9B is a light micrograph of a microparticle of the present invention illustrating internal voids within the microparticle, the particles being approximately 100 to 150 microns in diameter;

FIG. 10A is an SEM, imaged at 150× magnification, of an embodiment of microparticles of the present invention that are loaded with lidocaine and having a smooth particle surface;

FIG. 10B is an SEM, imaged at 500× magnification, of the microparticles of FIG. 10A;

FIG. 11A is an SEM illustrating craggy morphology of PVA foam particles, the particles being about 100 to 150 microns across;

FIG. 11B is an SEM illustrating the smooth, spherical morphology of one embodiment of a microparticle of the present invention, the particles being about 20 to 200 microns across;

FIG. 12A is an SEM, imaged at 150× magnification, of another embodiment of a microparticle of the present invention being lidocaine loaded and having microporous surface morphology;

FIG. 12B is an SEM, imaged at 500× magnification, of the embodiment of the present invention shown in FIG. 12A;

FIG. 13A is an SEM, imaged at 140× magnification, of a further embodiment of microparticles of the present invention, in this instance having a “brain-like” convoluted surface morphology;

FIG. 13B is an SEM, imaged at 500× magnification, of the embodiment of the present invention shown in FIG. 13A;

FIG. 14 is an enlarged photograph showing two embodiments of microspheres of the present invention in powder form;

FIG. 15 is a schematic representation of fibroids present in a human uterus;

FIG. 16 is a schematic representation of the apparatus of the present invention being used to perform a uterine fibroid embolization;

FIG. 17 is a schematic representation showing of the injection of embolic agent of the present invention into a human uterine artery;

FIG. 18A is a light micrograph, at about 20× magnification, showing injection of 10 μm microparticles of the present invention from a catheter;

FIG. 18B is a light micrograph, at about 20× magnification, showing injection of 80 μm microparticles of the present invention from a catheter;

FIG. 19 is a graph of one example of a dosing regime for a patient undergoing a uterine fibroid embolization (“UFE”);

FIG. 20 is a graph of normalized cumulative mass of lidocaine released from an embodiment of high lidocaine dose particles of the present invention;

FIG. 21A is a high performance liquid chromotography (HPLC) standard chromatogram for lidocaine dissolved in water;

FIG. 21B is a high performance liquid chromatography (HPLC) chromatogram for release of lidocaine eluted from an embodiment of microparticles of the present invention;

FIG. 22 is a structural formula illustrating the chemical process of the degradation of poly(latctic-co-glycolic acid) (“PLGA”) to lactic acid and glycolic acid;

FIG. 23A is a contrast angiogram of a renal cortex of a canine left kidney showing normal blood flow therethrough;

FIG. 23B is a contrast angiogram of the kidney of FIG. 23A showing selective catheterization of the cephalad pole of the kidney employing one embodiment of microparticles of the present invention;

FIG. 23C is a contrast angiogram of the kidney of FIG. 23B showing completed selective embolization of the kidney;

FIG. 24A is a contrast angiograph of a renal cortex of a canine left kidney showing normal blood flow therethrough;

FIG. 24B is a contrast angiogram of the kidney of FIG. 24A following embolization of the renal cortex using particles of the present invention having approximately a 80 μm external diameter;

FIG. 24C is a contrast angiogram of the kidney of FIG. 24B following embolization of the renal cortex using particles of the present invention having approximately 240 μm external diameter;

FIG. 25 is a flow diagram of examples of optional fabrication methodologies for manufacturing microparticles of the present invention;

FIG. 26 is a schematic representation of another embodiment of apparatus for in-vitro mechanism for testing of compression resistance of microparticies of the present invention;

FIG. 27 is a schematic representation of still another embodiment of apparatus for in-vitro mechanism for testing of compression resistance of microparticles of the present invention; and

FIG. 28 is a schematic representation of yet another embodiment of apparatus for in-vitro mechanism for testing of compression resistance of microparticles of the present invention.

### DETAILED DESCRIPTION OF THE INVENTION

The microparticles of the present invention (referred to herein as “inventive microparticles,” “microparticles,” “microspheres,” and “embolic agents”) may be employed in a wide variety of embodiments of varied characteristics and uses. Such microparticles can be resorbable or non-resorbable, and may be used for the transport of elutants and additives to desired locations in a patient. The microparticles are used in embodiments that cover a gamut of research, patient treatment and non-medical applications. For medical embodiments, microparticle characteristics include, but are not limited to, one or more of: ease-of-use; accuracy of delivery to target vessel(s), vascular beds or tissue(s); instigation and support of efficacious biological responses; and positive procedural outcomes for the patient. In many embodiments, the microparticles are catheter deliverable.

The figures and disclosure herein refer to characteristics of the microparticles. Certain observations which are disclosed are made by a human eye. Other observations are made under magnification through the use of instrumentation. Where images and observations are provided through the use of a light microscopy or scanning electric microscopy (“SEM”), the relevant magnification values are referred to as “X” or “times.”

The term “elution” is used herein to refer to any release of material from a microparticle. Materials typically provided for release include, but are not limited to bioactive agents, e.g., additives, coating materials, base polymer(s) or other material carried in, on, and/or with the microparticles. In usage, it may be stated in some embodiments that bioactive agents are “eluted” from a microsphere. “Elution rate” is one measure of the release or removal of any substance from a microsphere over time. Elutants from a microsphere can have elution rates which are constant or which vary over time and/or under changing conditions.

An “embolic agent” is a substance that is injected into a man-made or natural body lumen or cavity. Some embodiments of embolic agents obstruct the flow of blood or other liquids through a lumen or cavity. Some embodiments include characteristics of volume displacement, eliciting a biological response, and delivery of other agents or additives.

The term “bolus” is herein defined as a quantity, amount or number of microparticles. The term “bolus” may be used synonymously with dosage, quantity, treatment amount, and like terms which identify a quantity of microparticles, and particularly an amassed quantity of microparticles that are intended to be delivered together during a treatment procedure. A “bolus” of microparticles may be in powder form, suspended in vitro in a solution, or delivered or resident in vivo within a patient.

The term “fluid” herein is defined consistent with the context of the use or formation of the microparticles. In embodiments wherein the microparticles are contained, created, or in contact with a liquid phase, the term “fluid” refers to the solution or liquid phase in contact with the microparticle. For example, a bodily fluid includes all bodily liquids such as, and not limited to, blood, plasma, vitreous humor, interstitial fluid, air and intestinal or digestive fluids. A “target bodily fluid” includes any fluid of the body that is intended to interact with a microparticle. For example, in embodiments were the microparticles are injected into the bloodstream, the blood can be the target bodily fluid. When a microparticle is injected into the eye, the vitreous humor can be the target bodily fluid. The “target bodily fluid” is any bodily liquid that the practitioner desires to select for suspension of, or otherwise interaction with, a microparticle. In some applications, there can be more than one “target bodily fluid.” For embodiments not involving a body, the term “target fluid” is analogous and can include any fluid intended to suspend, come into contact with, or otherwise interact with a microparticle as desired by a practitioner. In the manufacture of the microparticles, “fluid” includes, but is not limited to, any liquid phase substance used in the manufacturing process, included in the microparticle, constituting the microparticle, or in contact with the microparticle. Other examples of liquids which herein are considered fluids are, but not limited to, injectate, carrier fluid, storage solution, base polymer solution, organic solvent solutions, aqueous solutions, water, contrast, contrast solution, and saline solution. The term “carrier fluid” includes any fluid (liquid or gas) which transports, or is intended to transport, a microparticle. For embodiments in which a microparticle is not in contact with a liquid, but is suspended or surrounded by a gas (e.g., air in an aerosol dispersion of microparticles), then “fluid” may include any gas(es) surrounding or within a microparticle.

The term “additive” broadly includes, but is not limited to, any substance added to a microparticle, microparticle coating, substances composing a microparticle (e.g., base polymers), substances in contact with a microparticle (solutions, liquid phases), and substances contained by a microparticle. “Additive” is a broad term including anything provided to a microparticle or to the constituents of a microparticle (e.g., base polymers, liquid phases, void volumes, coatings and any other constituent or substance of, in contact with, contained by, or interacting with, a microparticle) for any purpose.

Some abbreviations which are used throughout this application include:

° C.=degrees Centigrade

mm=millimeters

μm=micron

cc=cubic centimeters

ml=milliliters

g=grams

Throughout this disclosure, endpoints of ranges are considered to be definite and are understood to incorporate within their tolerance other values within the knowledge of a person having ordinary skill in the relevant arts. These other values include, but are not limited to, those which are insignificantly different from the respective endpoint as related to this invention. Endpoints are to be construed to incorporate values “about” or “close” or “near” to each respective endpoint. Range and ratio limits, recited herein, are combinable. For example, if ranges of 1-100 and 5-25 are recited for a particular parameter, unless stated otherwise it is understood that ranges of 1-5, 1-25, 5-100 and 25-100 are also contemplated.

Microparticles of the present invention can be fabricated from bioresorbable polymers (which term is intended to include polymers that are “resorbable,” capable of “resorption,” “bioabsorbable,” “absorbable,” or capable of “absorption”). The base polymer of a microparticle typically includes bioresorbable materials. Base polymers typically include one or more biocompatible materials that allow controlled bioresorption (i.e., “biodegradation,” “resorption,” “bioabsorbtion,” or “absorption”). The term “base polymer(s)” include polymers, copolymers and heteropolymers. Examples of base polymers include copolymers and homopolymers of poly[α-hydroxy esters]. Copolymers of poly[lactic-co-glycolic acid] (PLGA), Poly(glycolic acid) (PGA) and poly(lactic acid) (PLA) are included in this family of bioresorbable polymers. Copolymers of poly(lactic acid) and trimethylene carbonate (PLA-TMC), copolymers of poly(lactic-co-glycolic acid) and trimethylene carbonate (PLGA-TMC) are also used in some embodiments of microparticles. The above-identified copolymers do not require cross-linking. Some embodiments of microparticles have no cross-linking monomers or polymers at all. Other embodiments can have a degree of cross-linking based upon composition. Base polymers and microparticles utilizing a blend of non-cross-linked and cross-linked base polymers are encompassed in this invention. Any combination or mixture of base polymers herein disclosed may be employed in the production of microparticles of the present invention.

In some embodiments, microparticles can be formed through the precipitation of polymers from a base polymer solution to form aggregates which constitute a microparticle, or the microparticle's core, are in a coated or multi-layered microparticle embodiment (also referred to as “microparticle core,” “base polymer core,” “core”). A base polymer solution contains one or more base polymers dissolved in an organic solvent (such as, dichloromethane, chloroform, acetone, methylene chloride, ethyl acetate, etc). These polymers are referred to as “base polymer(s)” or “microparticle base polymer(s).”

Microparticles of the present invention typically have one or more base polymers. In coated or multi-layered embodiments, the base polymer(s) can be included in the core of the microparticle. Base polymers can include homopolymers of poly(α-hydroxy ester) which can optionally contain trimethylene carbonate. As discussed above, the base polymers are typically one or more of PLA, PGA, PLGA, PLA-TMC, PGA-TMC, PLGA-TMC, or other bioresorbable base polymers.

The base polymer, or mixture of base polymers, included in some embodiments of microparticles of the present invention are not cross-linked. Typically, the base polymers are linear chain polymers having an absence of cross-linking sufficient to allow partial or total resorption. Some microparticles have no cross-linking between polymer chains.

The base polymer of a microparticle may be composed of more than one type of monomer, polymer or substance. Such base polymer compositions are referred to as “mixed base polymer” and can include any combination of the base polymers disclosed herein.

In some embodiments, an implantable microparticle can have one or more additional layers referred to as a “coating” which is generally attached to, or supported by the microparticle. The coating in some embodiments is on the microparticle surface and surrounds a base polymer core. The coating of a microparticle may comprise one or more substances. Each coating layer is composed of substances that can be either pure or mixed. Coating substances may include, but are not limited to, gelatin, chitosan, hydrophilic polyurethane hydrogels, PLGA-PEG, PVA, collagen, chitin, albumin, alginate, polyethylene oxide, polyvinyl alcohols, pectin, amylose, fibrinogen and combinations thereof. More than one coating or layer can be used.

Other coating substances include, but are not limited to, organic and inorganic compounds and molecules, amino acids, proteins, enzymes, nucleic acid bases, bacteria, viruses, antibiotics, antibodies, antigens, prions, viruses, fats, nutrients, vitamins, elements, and mixtures thereof.

Coating substances can be employed to modify the release of one or more bioactive agents or additive(s), or to provide a surface for attachment of additional bioactive agents, additives or substances (such as drugs and/or antibodies) to the outside surface. Coatings can be bioactive agents or additives themselves. Coating(s) may also be employed to change the mechanical properties of the microparticle surface, e.g., the coefficient of friction, elastic modulus, priority, smoothness, or resistance to degradation. Coatings may be employed in time-release embodiments.

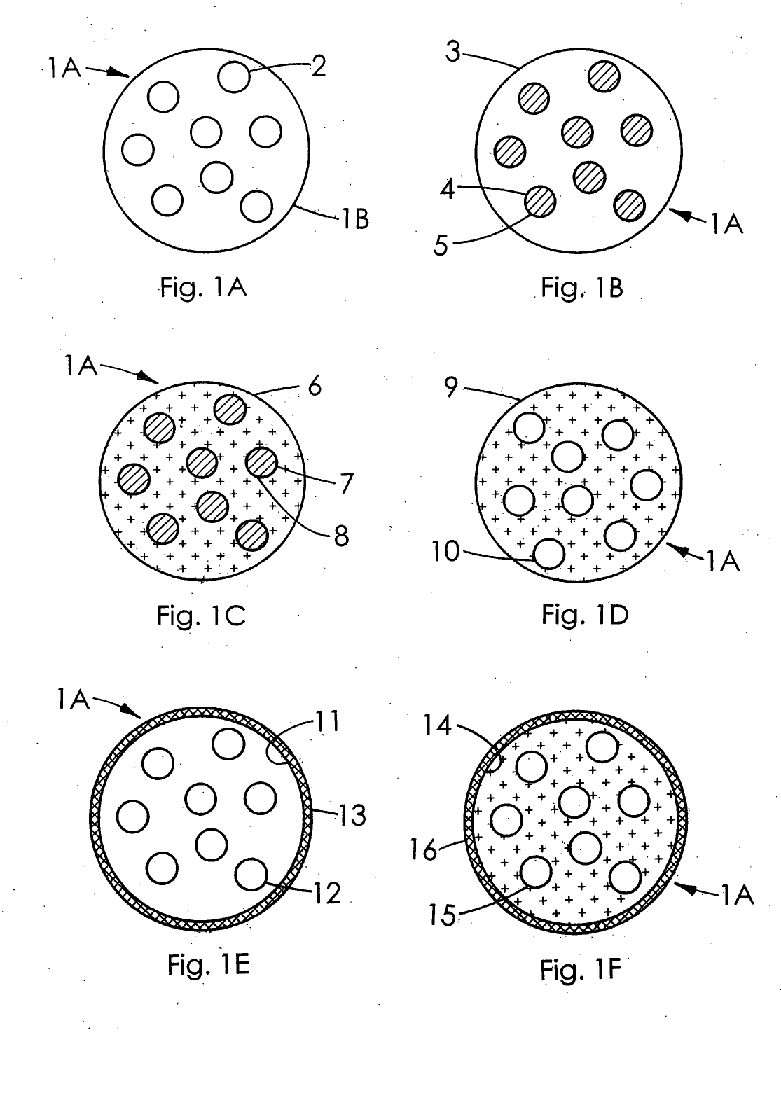

FIG. 1A illustrates a microparticle 1A of the present invention having a base polymer 1B and a void 2. FIG. 1B illustrates a microparticle 1A of the present invention having a base polymer 3 and a void 4 containing an elutant 5. FIG. 1C illustrates a microparticle 1A of the present invention having a mixed base polymer, or base polymer(s) mixed with elutant or additive(s) 6 and a void 7 containing an elutant 8, other polymer(s), additive(s) or mixture(s) thereof. FIG. 1D illustrates a microparticle 1A having a mixed microparticle base polymer, or microparticle base polymer(s) mixed with elutant(s) 9 or other additive(s) and a void 10. FIG. 1E illustrates a microparticle 1A having a microparticle base polymer 11, a void 12 and a coating 13. FIG. 1F illustrates a microparticle 1A having a mixed base polymer, or base polymer(s) mixed with elutant 14 or other additive(s), a void 15 which in some embodiments can contain one or more polymer(s), elutant(s), additive(s), or a mixture thereof, and a coating 16 including one or more polymer(s), elutant(s), additive(s) or mixtures thereof.

Materials for use as the inventive microparticles of the present invention employed in medical embodiments typically should be well-tolerated by patients and should be safe for use in the human body (e.g., cardiovascular system, or muscle-skeletal system). Target vasculatures can tolerate the presence of some embodiments of the microparticles without adverse biological sequella such as sustained, non-resolving inflammation. The microparticles of some embodiments promote efficacious biological responses.

Voids, inclusions, convolutions, additional materials and manufacturing factors (e.g., solution types, microparticle compositions, shear forces applied, and microparticle hardening) are used to engineer microparticle density. Density is engineered in some embodiments to achieve desired buoyancy values relative to target solutions or target bodily fluids, e.g., neutral buoyancy. Microparticles of the present invention can have an average density that is lower than that of the pure raw material base polymer(s) from which the microparticle is made. The ratio of base polymer density to non-elutant laden microsphere density is greater than 1.0 in some embodiments. In embodiments where the microparticle is laden with elutants the ratio of base polymer density to microparticle density can be either greater than or less than 1.0. Where the microparticle includes heavy elutant or additives, the ratio can be less than 1.0. Where lighter elutants or additives are used, the ratio can be greater than 1.0.

Employing empty voids reduces the overall mass of a microparticle and decreases particle density. In one embodiment, the utilization of voids and low-density material reduced the density of the microparticle to 40% less than the density of the utilized base polymer in its pure form under comparable conditions.

In one embodiment, microparticles are prepared where at least 95% of the microparticles have of a density greater than 0.9 g/cc but less than 1.4 g/cc. Microparticles in another embodiment have densities of 0.95 g/cc to 1.1 g/cc. In yet another embodiment microparticle density is approximately 1.0 g/cc. A typical microparticle of the present invention may have a density range of about 0.5 g/cc to about 2.00 g/cc, and more preferably between about 0.75 to about 1.5 g/cc, and more preferably between about 0.8 to about 1.4 g/cc.

Microparticle density may be manipulated during manufacturing, or modified by adding, substances to formed microparticles.

The specific gravity of a microparticle can be modified or engineered to have a desired value by manipulating the microparticle density. For many applications it is desirable to produce a microparticle that has a specific gravity similar to that of the solution in which the microparticles are injected or a target bodily fluid into which the microparticles are injected. A specific gravity of 1.0 as compared to an injection solution, a target bodily fluid, injected, or carrier fluid, is utilized for some embodiments. Other embodiments can have a specific gravity from 0.6 to 1.4, 0.75 to 2.0, or 0.6 to 1.4 of a target bodily fluid, or of a solution in which they are suspended.

Some embodiments of microparticles have specific gravities of 1.0 relative to a 50:50 mixture of an X-ray contrast medium (also “contrast,” “contrast solution,” “contrast agent solution”) and saline solution. One example of a contrast solution that may be employed with embodiments of microparticles is OMNIPAQUE™ iohexol (manufactured by Amersham Health, a division of Amersham PLC at 101 Carnegie Center, Princeton, N.J. 08540).

The engineering of density and specific gravity values are utilized to achieve buoyancy properties beneficial for microparticle use in biological or other systems. Manufacturing techniques disclosed herein encompass the production of embodiments of microparticles with buoyancies from 0% to 100% of the inherent buoyancy value of the microparticle's pure raw material form of base polymer(s). Microparticles having neutral buoyancy, or buoyancy values within 10% of the target bodily fluid into which the microparticles are injected, are utilized in some embodiments.

A buoyancy value approximating that of the carrier fluid injectate, a target bodily fluid increases suspension time in the carrier fluid (e.g., from 0-59 minutes, 1 or more hours, to 1 or more days, to 1 or more weeks, to 1 or more months, to 6 or more months, to 1 or more years in suspension). These buoyancy characteristics facilitate injection through low-profile catheters.

The microparticles can be prepared in some embodiments to have an approximately neutral buoyancy (i.e., “neutral gravity” is a specific gravity 1.0 relative to a given reference solution, liquid composition, or fluid at a desired temperature and pressure) relative to a solution in which they are suspended, a fluid system, a carrier fluid, a target fluid or target bodily fluid in which the microparticles are to be placed. In one embodiment, microparticle neutral buoyancy is achieved by preparing microparticles having a density of between 0.9 and 1.4 g/cc. Typically a specific gravity within 10% of an injectate is chosen (e.g., 50:50 saline solution to contrast agent solution).

Carrier solutions typically are composed of one or more liquids including alcohol, organic liquids, aqueous drug solutions, or any other aqueous and embolic agent compatible solutions. The fluids can serve as the solution in which the microparticles are suspended at the time of injection (e.g., saline solution, or a contrast agent solution) forming the injectate. In some embodiments, the liquids in which some embodiments are created, stored, transferred and prepared for use also can be utilized as carrier fluids.

Microparticles can typically be homogeneously suspended in a solution when the density of the microparticle to within 10% to 15%, or closer, to that of the solution in which they are suspended whether in vitro or in vivo. Embodiments of microparticles having densities within 10% to 15% do not readily separate from an injection solution (carrier solution) for periods of time having clinical relevance. Preferably microparticles of the present invention have densities between 5% to 15% to that of the solution.

Microparticles can be formed with or without voids. Microparticle void volume can range form 0% to 98%. The presence of voids, void fraction, and void distribution can be engineered characteristics of a microparticle. Factors which can be manipulated to affect void formation include base polymer composition, solution viscosity and the emulsion technique employed (e.g., whether single emulsion, or double emulsion, or multiple step processing).

A microparticle of any external diameter (e.g., from nanometers in scale up to 2000 microns, or larger) can contain empty voids, or voids which are filled with materials different from the predominant base polymer of the microparticle. Voids can form or can contain a different phase or type of material from the base polymer. Voids and filled voids can occupy up to 98% of total microparticle volume. The number, size, and concentration of void spaces can be controlled. The number of void spaces can range from a single void to thousands of voids or more. Void diameters can range from nanometers to one millimeter or more. Void volumes ranging 5-10%, 15-30% 40-60% of microparticle volumes are utilized in some embodiments. Void volumes of 5%, 20% and 45% of microparticle volume are typical.

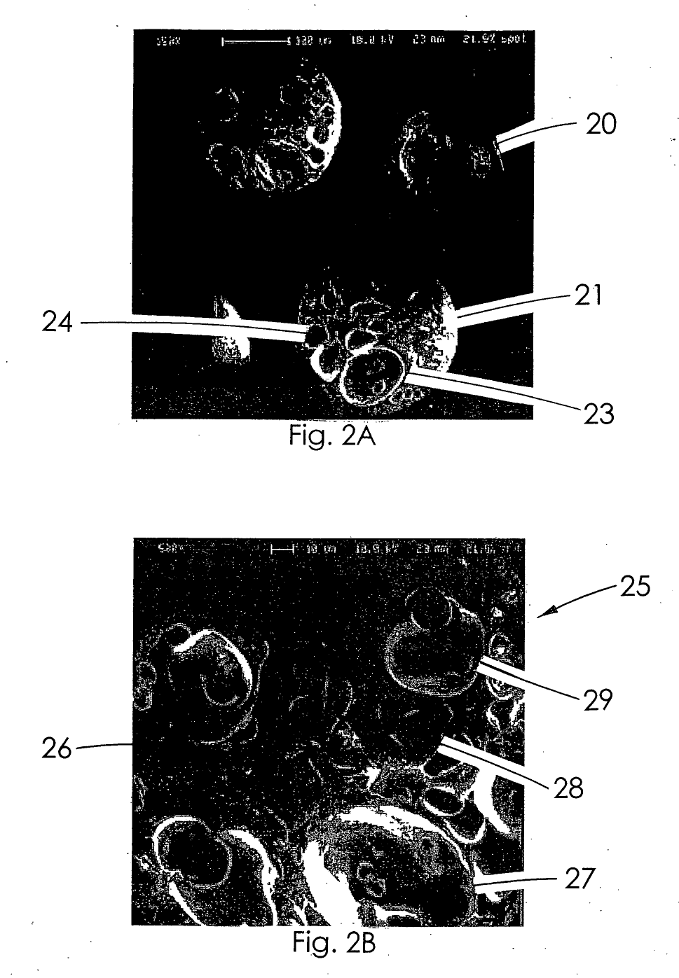

FIG. 2A is an SEM micrograph imaged at 150× of 20 wt % lidocaine-loaded microspheres having voids of various sizes 20 and including a base polymer 21. FIG. 2A also shows a microsphere having a large void 23 and a small void 24. FIG. 2B is an SEM micrograph imaged at 500× of a 20 wt % lidocaine-loaded microsphere 25 having voids of varying volumes and including a microparticle base polymer 26, a large void 27, a small void 28, and a medium void 29.

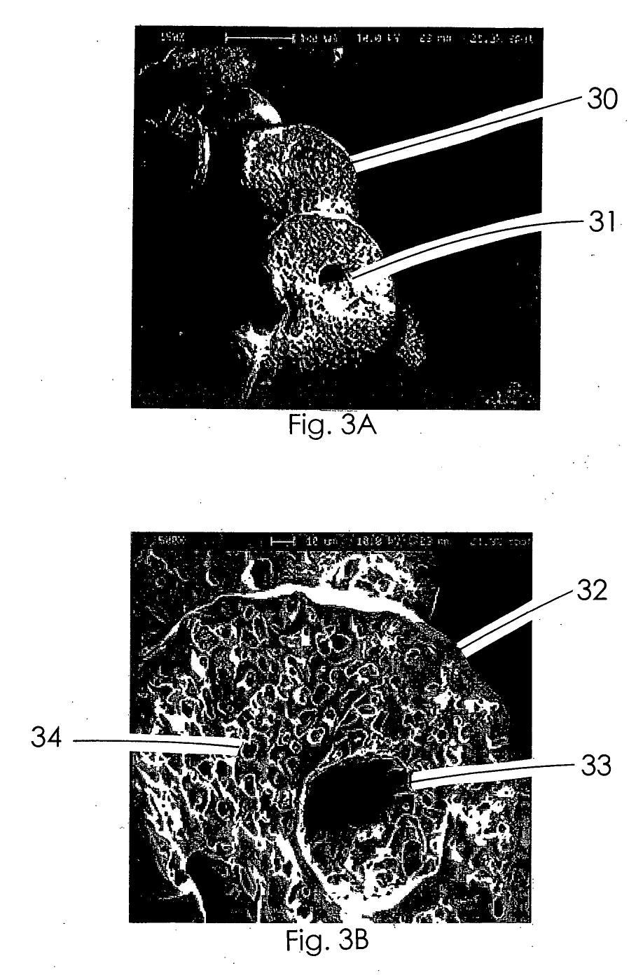

FIG. 3A is an SEM micrograph imaged at 150× of a lidocaine-loaded microparticle having voids 30 and another microparticle including a large void 31. FIG. 3B is an SEM micrograph imaged at 500× of a lidocaine-loaded microparticle having voids 32 and including a large void 33, and a small void 34.

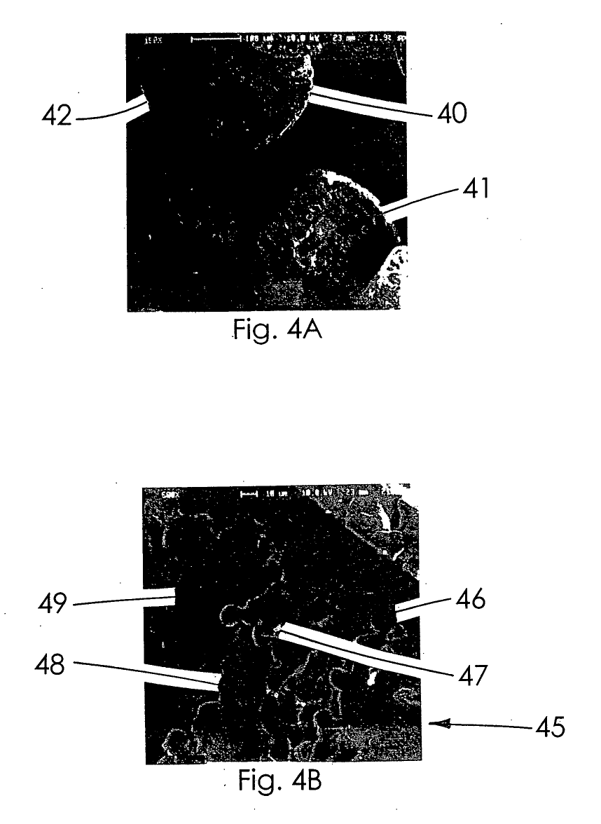

FIG. 4A is an SEM micrograph imaged at 150× of lidocaine-loaded microspheres having convolutions and internal voids of varying volumes and interconnectivity 40 and showing a microparticle base polymer 41 and a large void 42. FIG. 4B is an SEM micrograph imaged at 500× of a lidocaine-loaded microsphere 45 having convolutions and internal voids of varying volumes and interconnectivity showing a microparticle base polymer 46, an interconnection 47, a convolution 48, and a void 49.

The microparticles of the present invention may exhibit a lubricious/non-clogging characteristic (herein referred to as “lubriciousness”). Lubriciousness is attributable to low frictional properties of some microparticle embodiments and can be engineered by manipulating factors such as surface area, surface characteristics, elasticity, microparticle shape and the microparticle's constituent materials. For example, the hardness, hydrophobicity, or compression resistance associated with a microparticle base polymer or coating substance can affect lubriciousness of a microparticle. The composition of the microparticle, the nature of the elutants, internal structures and morphology of a microparticle can affect lubriciousness. Microparticles can be engineered to obtain uniform shape, constitute base polymers that are not tacky or adherent, or achieve a generally spherical shape. Each of these factors affects lubriciousness.

Lubriciousness may facilitate injectability and the catheter delivery of the microparticles. Lubriciousness can reduce, or eliminate, the clogging of a delivery catheter during microparticle injection. Microparticles of the present invention may be easily administered to patients through catheter injection. Lubricious microparticles reduce or eliminate the need for catheter flushing. Lubriciousness may enhance the performance of microparticles as embolic agents during both in vitro testing and in vivo tests such as canine kidney infarction procedures.

Microparticles of the present invention being delivered to a carrier solution exhibit very low requirements, or non-existent requirements, for a differential injection pressure that is above the injection pressure required for the same carrier fluid without microparticles. The pressure required for the injection of a carrier fluid having microparticles of the present invention through catheter equipment is for some embodiments not greater than, or minimally different from, the injection pressure required to administer the carrier fluid, alone without microparticles, to a patient. Some embodiments have injection pressures for a carrier fluid having microparticles that are within 10%. Typically, differential injection pressure is less than 1 atmosphere greater than the injection pressure of the carrier fluid alone. Near zero percent differences in differential injection pressure may be achieved in some embodiments of the present invention.

The inventive microparticles of some embodiments exhibit non-aggregability within a target vasculature. Non-aggregability is a characteristic that may be consistent with lubriciousness.

Microparticles exhibit structural stability, strength and resistance to fracture. The microparticles of some embodiments of the present inveniton are able to maintain their structure and strength sufficiently to function as effective embolic agents even in high-pressure systems or hypertensive circulatory systems. Microparticle stability is typically sufficient to maintain stability until the manifestation of desired biological effects of embolization or treatment (e.g., mechanical blockage, or occlusion, of a vessel and the completion of a fibroplastic response) with the microparticle. Embodiments directed toward drug delivery microparticles can be produced where the resorption rate exceeds, for example, the time required to deliver elutants, or to induce a chronic reduction of tumor symptomology. Structural stability is considered to exist, or is maintained, if the microparticle is capable of functioning in its intended therapeutic or embolic function for any amount of time. The microparticles can be engineered for resorption after hours, days or years. Typically microparticle structural stability may be maintained to about 5 years, and more preferably between about 30 days to 180 days, or about 30 days to 90 days.

It is believed that it is desirable for some embodiments of microparticles of the present invention to exhibit compression resistance, even in the presence of having voids or other internal structures. Compression resistance is defined as the ability of a microparticle to resist deformation without fracture, or to resist a pre-defined degree of a dimensional change when an external load is applied. For compression resistant microparticles of the present invention, an original microparticle shape can be maintained to an engineered tolerance. In some embodiments, if the tolerance to deformation is exceeded, the microparticle can fracture. External physical loads of 0.1, 0.03, or 0.07 kilograms are resisted by certain embodiments of the microparticles of the present invention. Microparticles compression resistant to external physical loads of up to 0.2 kilograms or more can be utilized in the present invention. In some embodiments, microparticles having an original external diameter, resist a deformation which changes the original external diameter by values selected from 0 to 30%. If deformation exceeds the desired value, the microparticle can fracture. For example, in one embodiment, a compression resistant microparticle resists an external physical load of 0.2 kg. If the physical load exceeds 0.2 kg, then the microparticle may fracture. Some embodiments of microparticles can exhibit a resistance from, for example, about 0% through 20% deformation of their original external diameter (and their mathematically analogous geometric displacement or change) from extrinsic physiological loads of less than 0.1 kilograms.

In some embodiments the base polymers of the microparticles themselves exhibit compression resistance.

This invention encompasses a new technique for measuring the compression resistance of microparticles. The process for measuring compression resistance includes providing microparticles in a carrier solution and passing those microparticles through a cylindrical test channel of specified internal diameter. The internal diameter can range from nanometers to millimeters and is selected based upon the degree of compressed resistance that is sought. A compression resistant microparticle will not significantly fracture or deform, e.g., a less than 10% deformation of original average microparticle external diameter as it passes through the test channel can be considered compression resistant. A microparticle which is not compression resistant will fracture, break or deform to a significant degree, such as by greater than 20% deformation of original average microparticle external diameter.

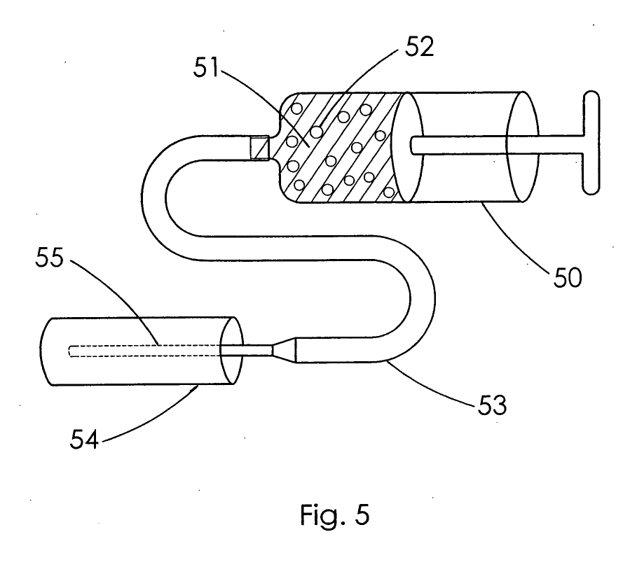

FIG. 5 illustrates one compression test apparatus for determining microparticle compression resistance within the present invention. FIG. 5 depicts a syringe 50 containing a carrier solution 51 with a microparticle 52, a tube of first internal diameter (D1) 53, a test channel having second internal diameter (D2) 55, and a test cylinder 54. The average external diameter of the microparticle and D2 can be of any ratio necessary to measure a given degree of compression resistance.

In one embodiment, a microparticle is considered to be compression resistant if it can not be injected undamaged through a rigid conduit having an internal diameter D2 that is 10% smaller than the external diameter of the microparticle. In another embodiment, a microparticle is considered to be compression resistant if it can not be injected undamaged through a rigid conduit having an internal diameter 20% smaller than an external diameter of the microparticle. In yet another embodiment, a microparticle is considered to be compression resistant if it can not be injected undamaged through a rigid conduit having an internal diameter 30% smaller than an external diameter of the microparticle.

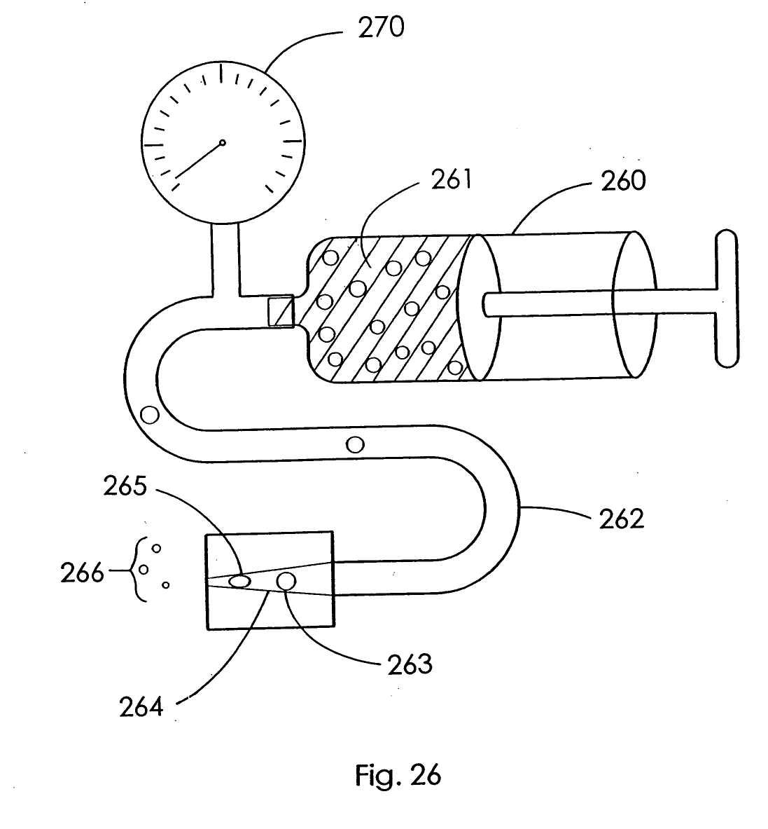

FIG. 26 illustrates another embodiment of an in-vitro mechanism of testing for compression resistance. A syringe 260 filled with microparticles suspended in a carrier solution forming an injectate 261 is injected into tube 262 with a pressure gauge 270. The microparticles travel through tube 262 and a single microparticle 263 will enter the test channel 264. This microparticle is required to compress in order to travel down the tapered test channel 264. Under very little pressure (e.g., 0.1 psi) the microparticle is pressed into the tapered channel at a point that matches the microparticle external diameter. As further back pressure is applied, the microparticle can deform to move down the tapered channel. The taper is well defined geometrically and any distance moved from the original matched diameter point allows for determination of compression resistance and is a function of back pressure applied. If enough back pressure is applied, the compression resistant microparticle may fracture 266 and pass out of the test channel 264. A microparticle that is not compression resistant 265 may grossly deform as it travels down the channel.

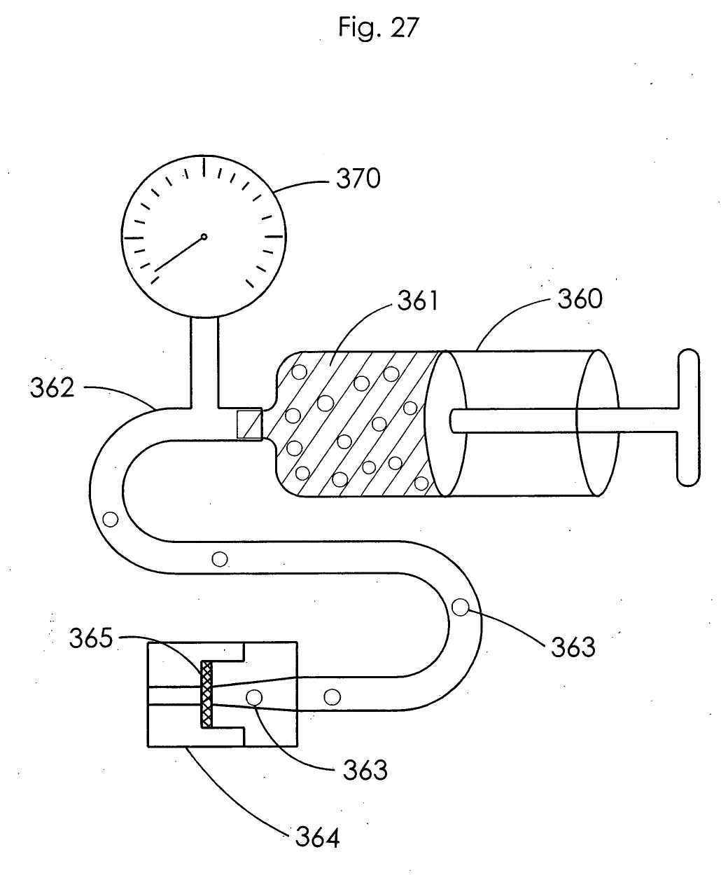

FIG. 27 illustrates another embodiment of an in-vitro mechanism of testing for compression resistance. A syringe 360 filled with microparticles suspended in a carrier solution forming an injectate 361 is injected into tube 362 with a pressure gauge 370. The microparticles 363 travel through tube 362 and enter a filter holder 364 containing a filter screen 365 with openings smaller than the diameter of the microparticles. The microparticles 363 are required to compress in order to pass through the filter screen. A microparticle that is not compression resistant may grossly deform, especially under back pressure, allowing it to pass through the filter screen. A microparticle that is compression resistant will not pass through the filter screen unless enough back pressure is applied to cause the compression resistant microparticle to fracture and allows fractured pieces to pass through the filter screen 365.

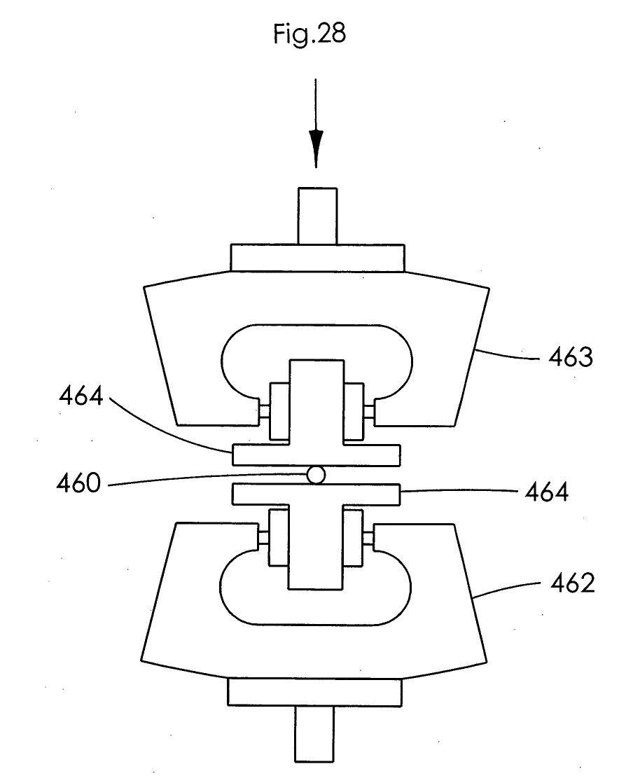

FIG. 28 illustrates another embodiment of an in-vitro mechanism of testing for compression resistance. A compression tester may be constructed to apply compressive force to a microparticle 460 and to simultaneously measure any measurable strain via jaw movement. For instance, the microparticle may be placed between two jaws 462, 463 that are connected to the compression tester. The lower jaw 462 is fixed in place and the upper jaw 463 is movable and connected to a load cell that can measure the amount of force applied by the jaw to a test specimen. The jaws hold platforms 464 that secure the microparticle 460 in place. The compression tester applies a measured force to the microparticle by moving the top jaw 463 so as to compress the microparticle. The displacement of the jaw 463 is simultaneously measured to determine any deformation of the microparticle at the applied load. Increasing forces are applied to the microparticle 460 until it fractures. In one embodiment, the diameter of a compression resistant microparticle will deform less than about 30% before fracturing. In another embodiment the diameter of a compression resistant microparticle will deform less than about 25% before fracturing. In another embodiment the diameter of a compression resistant microparticle will deform less than 20% before fracturing. In other embodiments, the microparticles may deform less than about 15%, 10%, or 5%.

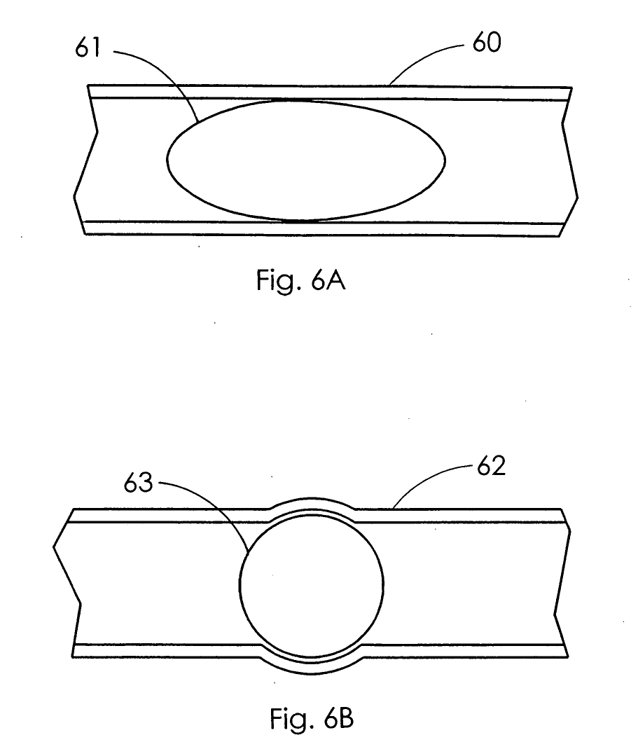

FIG. 6A illustrates a blood vessel 60 holding a compressible microsphere 61. As can be seen, due to the compressive nature of the microsphere 61, the blood vessel is not deformed despite the fact that the blood vessel has an inner diameter smaller than the outer diameter of the uncompressed microsphere 61. A compressible microparticle will travel to a distance into a blood vessel where an equilibrium point is reached where the outward force exerted by the compressible microparticle is counteracted by the restricting, force imposed by the vessel wall. Unfortunately, this position of equilibrium can be difficult to predict since it can be significantly altered by a number of parameters that are quite variable, including for instance, wide ranging blood pressures.

Compression resistant embodiments of the present invention do not significantly deform during travel through a blood vessel. In one embodiment the change in microparticle external diameter resulting from compressions was about zero. In other embodiment the change in microparticle external diameter was less than 25%. FIG. 6B illustrates a blood vessel showing deformation 62 holding a compression resistant microsphere 63. When compression resistant embodiments of microparticles are utilized the blood vessel may deform to accommodate the presence of the microsphere (as illustrated in FIG. 6B). The compression resistant microparticle will travel to a distance into the blood vessel where an equilibrium point is reached between the inward force exerted by the compliant vessel wall and the outward resisting force exerted by the microparticle. It has been determined that by using non-compressive microparticles of the present invention, microparticle performance can be more readily predicted prior to injection and successful microparticle pre-selection can be more easily achieved.

Compression resistant microparticles allow for accurate size matching to the targeted vessels or tissues. Size-matching between a compression resistant microsphere and a target blood vessel internal diameter includes microspheres having average external diameters ranging from 0% to 25% (or more) different (larger or smaller) from the vessel internal diameter. Flexibility in matching exists because the microparticle does not significantly change shape as it travels through the vasculature while the internal diameter or shape of the vessels encountered may be changed or deformed by the presence of the microparticle. The vasculature typically deforms to accommodate one or more microparticles.

The microparticles can experience lodgment in a vessel or remain free-floating. “Lodgment” occurs when a microparticles rate of movement through a system (i.e., velocity) approaches zero, or is zero.

Microparticles of the present invention are adapted to have a contact surface area during lodgment in a vessel or tissue from 0.025% to 90% (prior to fibroplastic response) of the external microparticle surface between the embolic agent and host vessel wall or embolized cavity or body spore, which may help facilitate elution and/or uptake of eluted drugs. During or after fibroplastic response, the entire lodged microparticle can be entirely surrounded by tissue (i.e. up to 100% microparticle external surface area contact) prior to and during resorption. Some embodiments of compression resistant microparticles are not susceptible to dislodgment associated with changes in vessel internal diameter (e.g., accompanying alterations in vessel tone such as vasodilation) or intraluminal pressure.

The resistance to extrinsic compression exhibited by the microspheres of the present invention improves their ability to create a durable embolization. In some embodiments the microspheres achieve an effective seal (up to 100% seal) of the surrounding vessel against fluid (blood) flow. This can result in complete, or nearly complete, blood occlusion. A strong seal can almost eliminate the possibility of vessel recanalization.

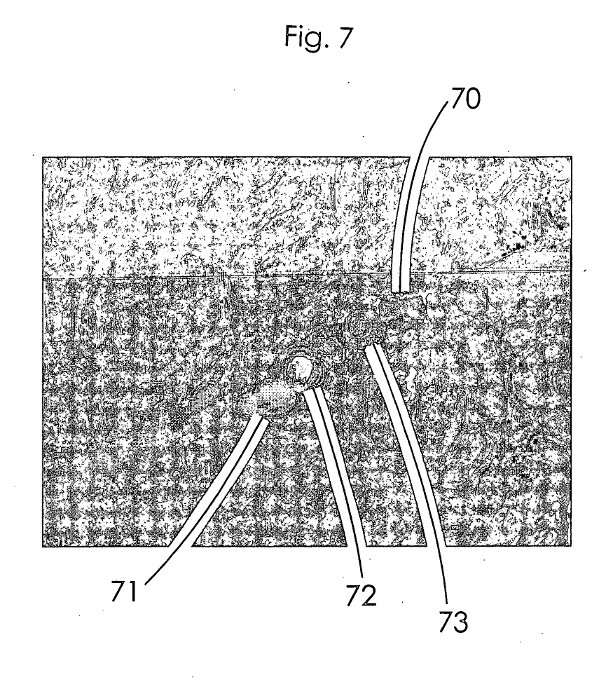

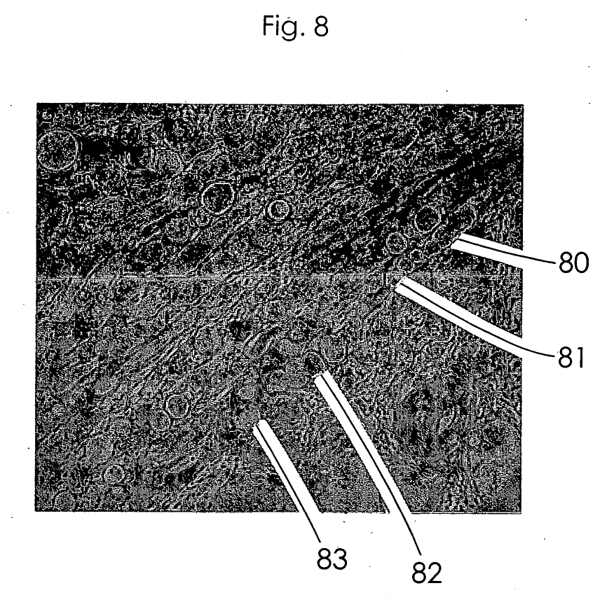

These potential benefits are evident upon review of histology collected from animals injected with microparticles of the present invention. FIGS. 7 and 8 are images of microparticles of the present invention embolizing vasculature. FIG. 7 is a pictomicrograph of a blood vessel 70 in which microparticle 71, microparticle 72, and microparticle 73 have been deployed. FIG. 8 is a pictomicrograph of a blood vessel 80 holding microparticle 81 and tissue in which microparticle 82 and microparticle 83 have been deployed. Evident in FIG. 7 and FIG. 8 is the deformation of the vessel wall to accommodate the presence of the incompressible microspheres and lodgment.

Microparticles of the present invention can be designed to have a wide variety of structures and compositions. Microparticle characteristics which may be adapted, modified and designed include, but are not limited to composition, density, void properties (e.g., void fraction, void volume, void size), base polymer composition, additives and agents, coating, size, surface area, surface topography texture, porosity, convolutions fissures, hardness, lubriciousness, strength, compression resistance, porosity, decomposition and resorption characteristics.

Examples of the internal structure of microparticles of the present invention are presented in FIGS. 2A, 2B, 3A, 3B, 4A, 4B, 9A, and 9B.

A microparticle embodiment which is used as an embolic agent can be created, or selected, to posses a particular geometry. The microparticles of the present invention can have an irregular or spherical configuration. For spherically shaped embodiments, a microparticle is typically referred to as “microsphere.” The process used to fabricate a microparticle of the micrometer range can also be employed to produce microparticles embodiments having external diameters in the nanometer range (i.e., “nanoparticles”, or “nanospheres”). Nanoparticles find application in targeting, for instance, phagocytic cells. These target cells can be macrophages. Typically, microparticles or microspheres have a well-characterized primary dimension which can be used to match a given application or vasculature. The microparticle average external diameter in some embodiments is an example of a primary dimension. Precise vessel targeting is achieved by matching a microparticle geometry (e.g., average external diameter) and target vessel dimensions. Generally, the smaller the microparticle, the narrower the vessel which can be treated. Microparticles can be prepared with external diameters from 20 nanometers to 5 mm. In some embodiments smaller microparticles from 25-200 nanometers external diameter are used. A clinically effective bolus of microparticles prepared for catheter injection in some embodiments has an average microparticle external diameter of greater than 10 microns. In one embodiment, at least 95% of the microparticles have an external diameter of greater than 10 microns for use in catheter injection.

The average external diameter of a microparticle is in-part dependent upon the fluid viscosity of the emulsion of base polymer and continuous phase. A more viscous solution will yield larger microparticles upon applying shear forces during microparticle formation. The less viscous the emulsion of polymer in solution, the smaller the resulting microparticles produced by a given shear force.

The shear force introduced into the manufacturing process, affects microparticle size. Greater shear forces introduced into the system produce smaller microparticles, or even nanoparticles.

Apart from controlling intrinsic properties and processing variables, microparticles of a specified external diameter may also be obtained by sieving previously prepared microparticles. A bolus of microparticles having a desired external diameter distribution may be obtained by sieving microparticles from different batches of microparticles. A combination of techniques such as intrinsic factors, manufacturing process and sieving may be utilized to prepare a bolus or microparticle of a desired range of external diameter. The external diameters of microparticles to be tightly controlled to exact dimensions. Control of external diameter selection through sieving can be exact (i.e., 0% difference), and is typically to within 50% (larger or smaller) of target external diameter. In some embodiments sieving is conducted after a hardening step.

Microparticles of-the present invention can be created with external diameters that range between approximately 10-2000 μm for embolization purposes. Typical ranges of external diameters of microspheres include about 40-120, about 100-300, about 300-500, about 500-700, about 700-900, and about 900-1200 microns. In one embodiment microparticles having an external diameter of about 2000 microns is achieved. Sieved microparticles and manufactured microparticles of a desired size can be combined as desired.

Microparticles, including microparticles in the nanoparticle size range, can be used as general free blood circulating agents functioning as drug-delivery vehicles. They can be administered via direct injection into the bloodstream or tissue. Microparticles (including nanoparticles) can also be used as tissue bulkers via direct injection into tissue masses or supplied through the vasculature.

Microparticles of the present invention can be engineered to have a desired shape, geometry and surface topography. Embodiments of the present invention include, but are not limited to, smooth spheres, pitted spheres, convoluted spheres, irregular shapes, shapes affected by additives and elutants, and shapes engineered to change or become modified during use or resorption.

One embodiment of the microparticles of the present invention has a generally uniform, smooth, spherical configuration when viewed at magnification levels up to 500× (500 times) by scanning electron microscope.

FIGS. 2A, 2B, 3A, 3B, 4A, 4B, 9A, 9B, 10A, 10B, 11B, 12A, 12B, 13A, 13B and 14 provide images of microsphere embodiments.

The surface area of microparticles of the present invention can be engineered, adapted, and modified. Microparticles can have a smooth spherical shell, or a textured surface providing an increased surface area in comparison to embodiments with a smooth surface of comparable average external diameter. Microspheres can exhibit a textured surface having pores, roughness, pitted features, convolutions, fissures, or porous surfaces. Other embodiments exhibit porous involutions. Surface features of a microparticle can appear exclusively as one type or can be of mixed types. Surface features may be predominant (i.e., more than 50% of surface), or minority (i.e., less than 50% of surface), or mixed in presence. The diverse surface topographies and textures can be observed via surface examination at an SEM magnification of 20-500×. The ratio of the surface area of a non-smooth particle to a smooth particle is typically greater than 1.0. In one embodiment, the microparticle surface provides a surface area that is up to 25% greater than that of a smooth spherical particle of comparable average external diameter. In other embodiments the surface area of the microparticle is at least 50%, 75%, or 100% greater than that of a smooth spherical particle of comparable average external diameter. In some embodiments the increased surface area provides an increased degradation rate in contrast to a smooth spherical particle of the comparable average external diameter and construction. In some embodiments the increased surface area provides improved tissue incorporation over a smooth spherical particle of the comparable average external diameter and construction. Some embodiments have surface areas hundreds or thousands of times greater than the surface areas of a comparable, or diametrically equivalent, smooth embodiments.

Further, in some embodiments of the present invention surface features may change over time as a function of the state of the microparticle and the environmental conditions to which the microparticle is exposed. In other embodiments, the surface features are generally constant and do not change significantly prior to any resorption process which may occur. Generally, resorption affects the surface features of a microsphere.

Microparticles of the present invention can be adapted to exhibit an increased surface area providing an increased elution profile of a bioactive agent as compared to the elution profile of a smooth spherical particle of comparable average external diameter and construction.

The viscosity (polymer/solvent ratio) of the polymer-based solution used for microparticle manufacture is a factor affecting the surface topography of the final microparticle. Base polymer solutions with lower viscosities provide microparticles with smoother surfaces, as compared to higher viscosity polymer based solutions which provide less smooth, high surface area, or “brain-like” convoluted topographies. The brain-like surface topography exhibited in some microparticles results from open spaces on the surface of the microparticle, which are a result of fissures which form between aggregate polymer chains.

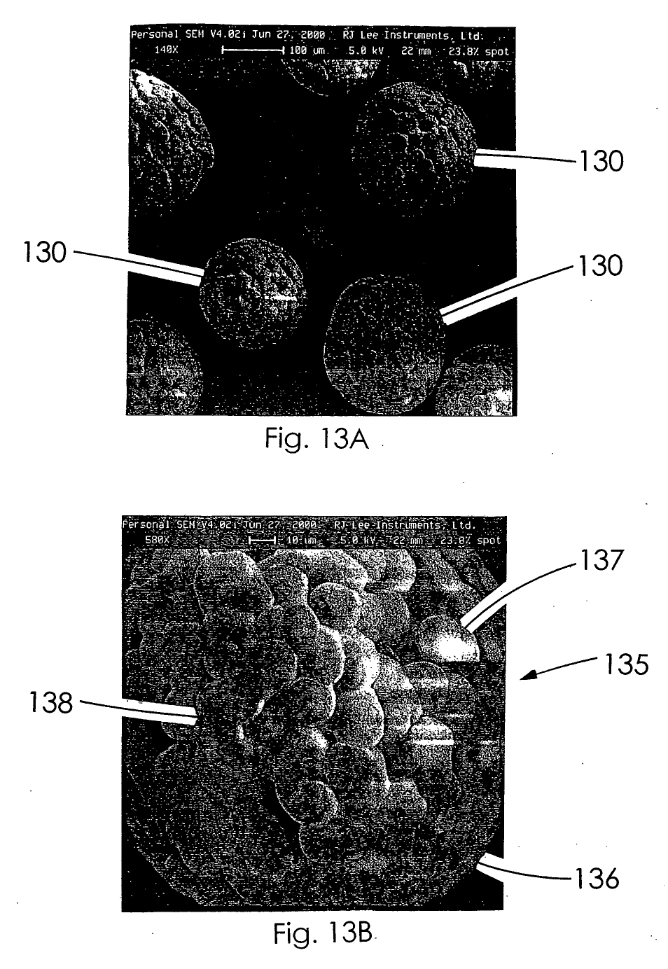

FIG. 13A and FIG. 13B are scanning electron micrographics (SEMs) of whole microspheres of the present invention, the outer surface has a distinct brain-like texture while maintaining an overall spherical shape. The base polymer solution from which brain-like topographies are achieved is an organic-based viscous polymer solution.

The surface topography of a microparticle can be engineered by varying the viscosity of the base polymer solution, aqueous or organic phase characteristics, the shear forces applied during formation, elutant characteristics and coating properties. This results in what is referred to as engineered surface topography.

Example 16 shows the effect of solution concentration and viscosity on topography.

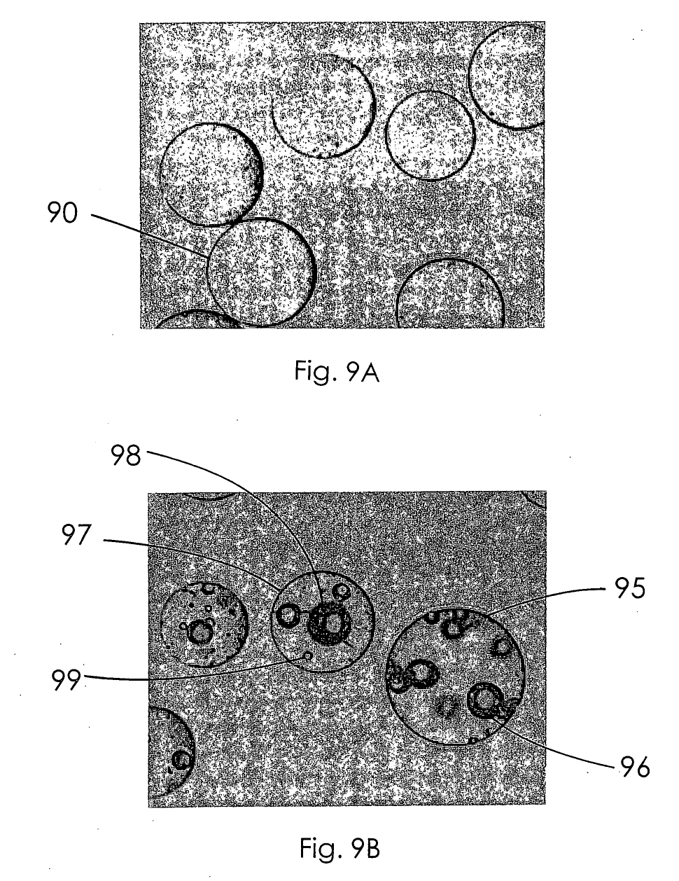

FIGS. 9A and 9B are micrographs of microspheres of the present invention with comparable surface topography but distinctly different internal structures. Notice that in FIG. 9B, the presence of void fractions and the absence of void fractions in FIG. 9A.

It is possible in some embodiments of the invention to maintain a consistent surface topography, while varying internal microsphere structure as shown in FIGS. 9A and 9B. FIG. 9A is a light micrograph of microparticle without voids 90. FIG. 9B is a light micrograph of microparticle with external diameters comparable to those of 9A and having voids 95, having a large void 96. FIG. 9B includes a light micrograph of microparticle with voids 97, having a large void 98 and a small void 99.

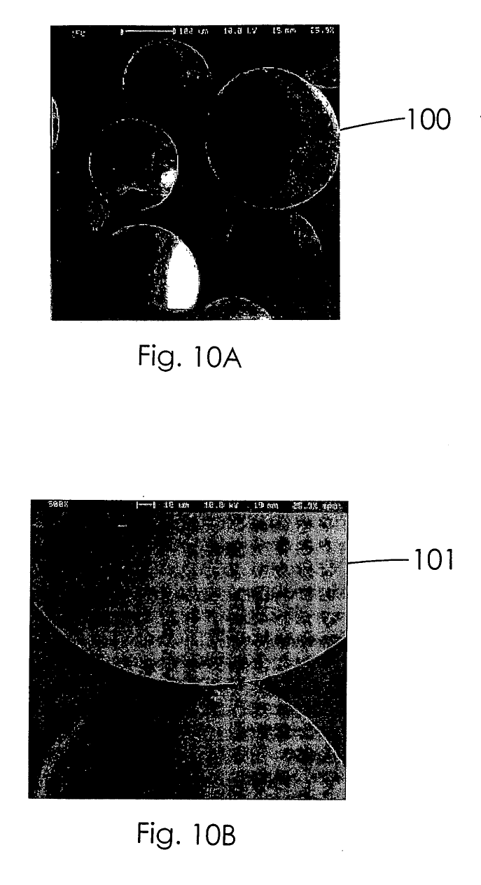

FIG. 10A is an SEM micrograph imaged at 150× of microspheres of the present invention with smooth surfaces 100. FIG. 10B is an SEM micrograph imaged at 500× of these microspheres with smooth surfaces 101.

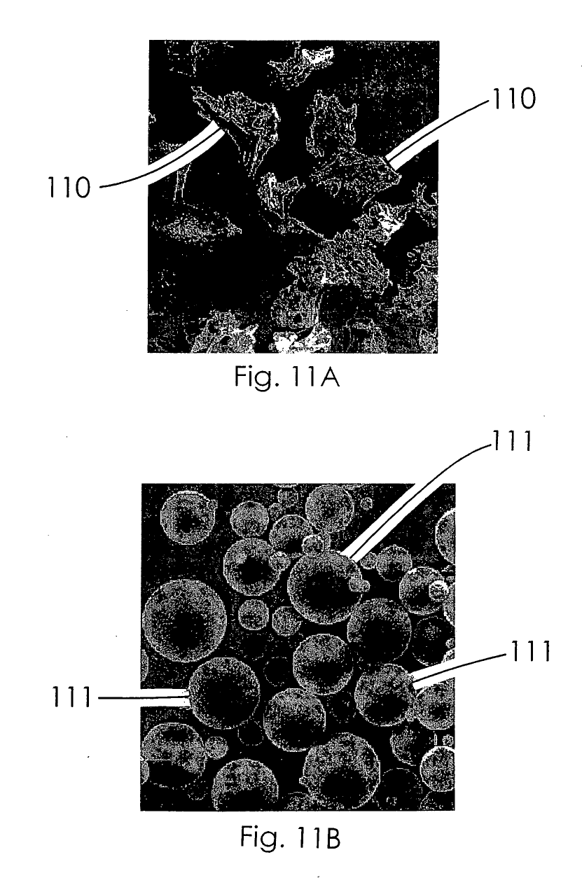

FIG. 11A is an image of PVA foam particles (150-250 μm external diameter particle size) 110. FIG. 11B is an image of microspheres of the present invention (10-250 μm external diameter microsphere size) 111.

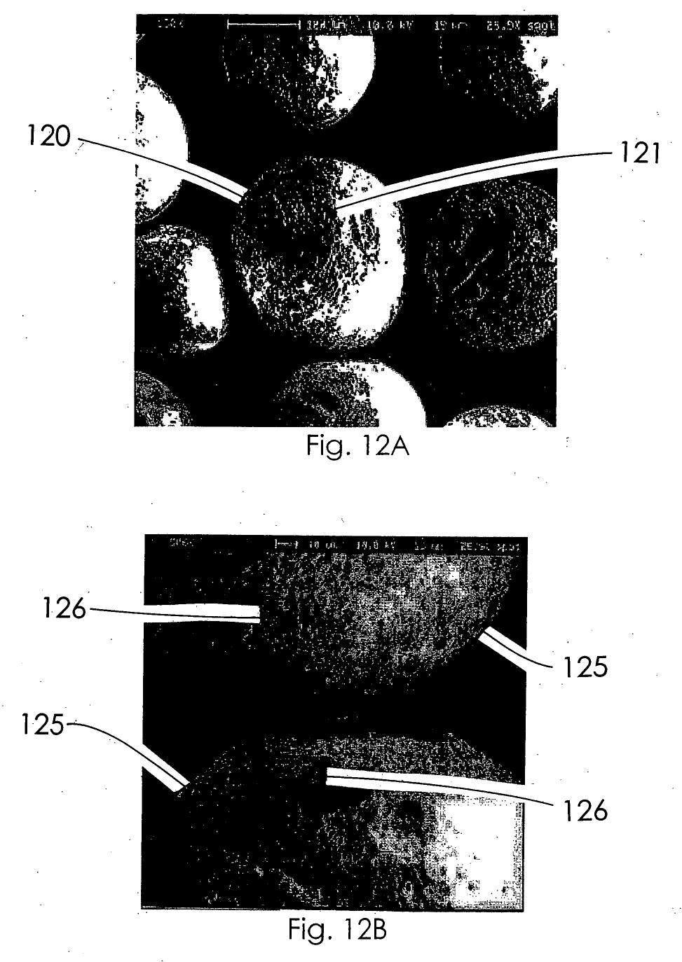

FIG. 12A is an SEM micrograph imaged at 150× of lidocaine-loaded microspheres having microporous surface 120. FIG. 12A also shows lidocaine-loaded microsphere having microporous surface 121. FIG. 12B is an SEM micrograph imaged at 500× of lidocaine-loaded microspheres having microporous surface 125, also showing a micropore of a lidocaine-loaded microsphere 126.

FIG. 13A is an SEM micrograph imaged at 140× of microspheres of the present invention having convolutions and convoluted or “brain-like” surface 130. FIG. 13B is an SEM micrograph imaged at 500× of microsphere having convolutions and a convoluted or “brain-like” surface 135, including a microparticle base polymer 136, a small convolution 137, and a large convolution 138.

Hardness is a further characteristic that may be engineered within the present invention. Hardness is in part dependent upon the nature of the base-polymer, coatings, elutants, additives, and microparticle manufacturing and processing.

The hardening phase of microparticle formation is optional and organics or other included substances can be removed from the microspheres through liquid-liquid extraction techniques. Hardening can be accomplished through the use of a variety of solvents including organic solvents, aqueous solvents, or mixtures of different solvents.

In some embodiments, microparticles, are produced and prepared for use in a powder form. The powders are relatively free-flowing under ambient conditions. This characteristic may be observed in some embodiments by gently shaking a particle filled vial and noting the free flowing movement of the microparticles.

Microparticle powders may be contained or stored in a single use, sterile vial. The microparticles of the powders may be placed in solution with procedural techniques including, but not limited to, suspension in a fluid. The microparticles of the present invention can be used in the clinical indications. These re-suspended microparticles and injected through a catheter or applied directly into a tissue bed.

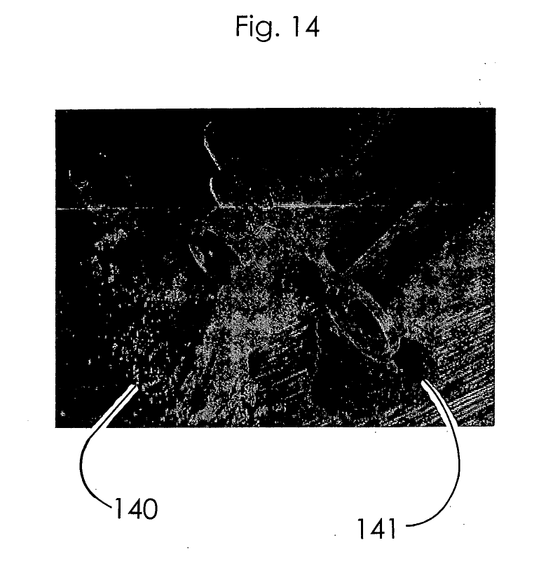

FIG. 14 is a photograph of drug eluting microspheres 140 of the present invention, and radio opaque (also known as “radiopaque”) microspheres 141 of the present invention.

Microparticles of the present invention can be injected through a small internal diameter infusion catheter. Microparticles of the present invention can also be injected through conventional, low profile infusion catheters. Embodiments of microparticles that are typically selected for catheter delivery are compression-resistant and comprise a non-cross-linked bio-resorbable material.

An embolic agent is a substance used to mechanically obstruct blood flow through a vascular conduit. Microparticles of the present invention can be utilized as embolic agents. The methods of the invention allow targeted delivery of embolic agents to the intended site of embolization to provide mechanical obstruction of blood flow. A bolus of microparticles of the present invention can be adapted for delivery through a catheter by means such as selecting desired external diameters, lubriciousness, compression resistance, density, buoyancy, coatings, and any other characteristic affecting injectability. Mechanical blockage of target vasculature can be achieved in some embodiments by embolization with one or more microparticles.

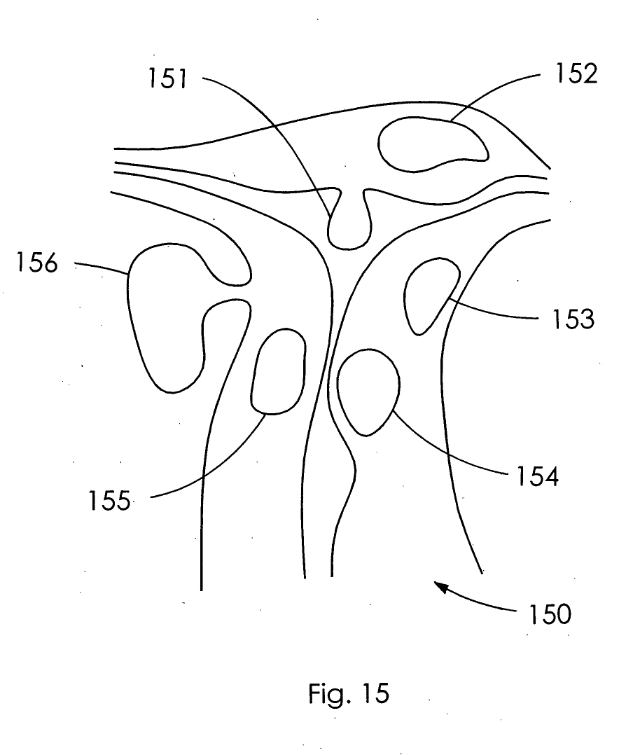

FIG. 15 is an illustration of a human uterus 150 showing fibroid tumors including a pedunculated submucosal fibroid tumor 151, an intramural fibroid tumor 152, a subserosal fibroid tumor 153, a submucosal fibroid tumor 154, an intramural fibroid tumor 155, and a pedunculated subserosal fibroid tumor 156.

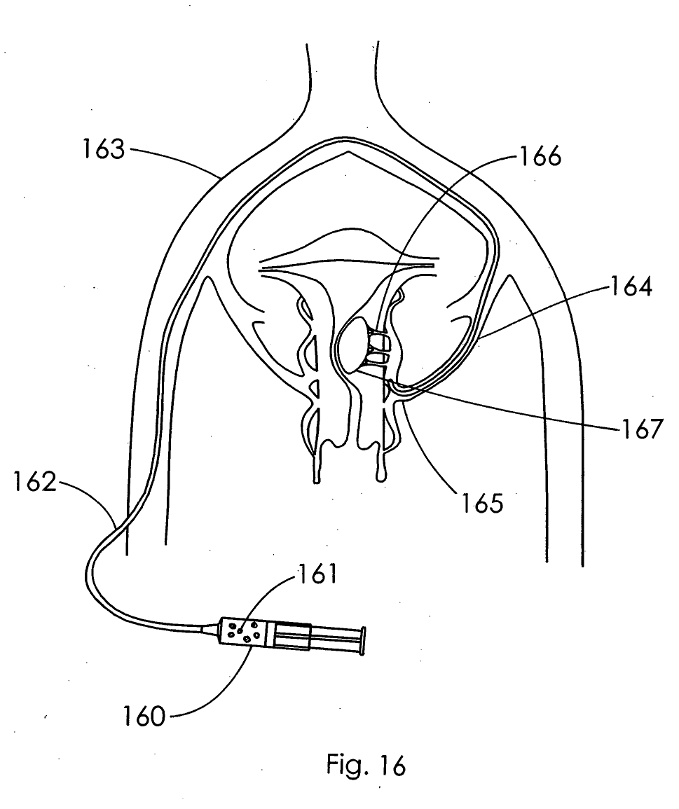

In one embodiment of the present invention, a catheter is guided angiographically to the uterine artery site. A pre-filled syringe with microparticles is then injected into the uterine artery to infarct the uterine fibroid. FIG. 16 illustrates the delivery of microparticles to a human uterus through the use of a catheter system including a syringe 160, a carrier solution with microparticles 161, and a catheter 162. The catheter is passed through the femoral artery 163 and uterine artery 164 to a location near the uterus 165 in which a fibroid tumor vasculature 166 feeds a fibroid tumor 167.

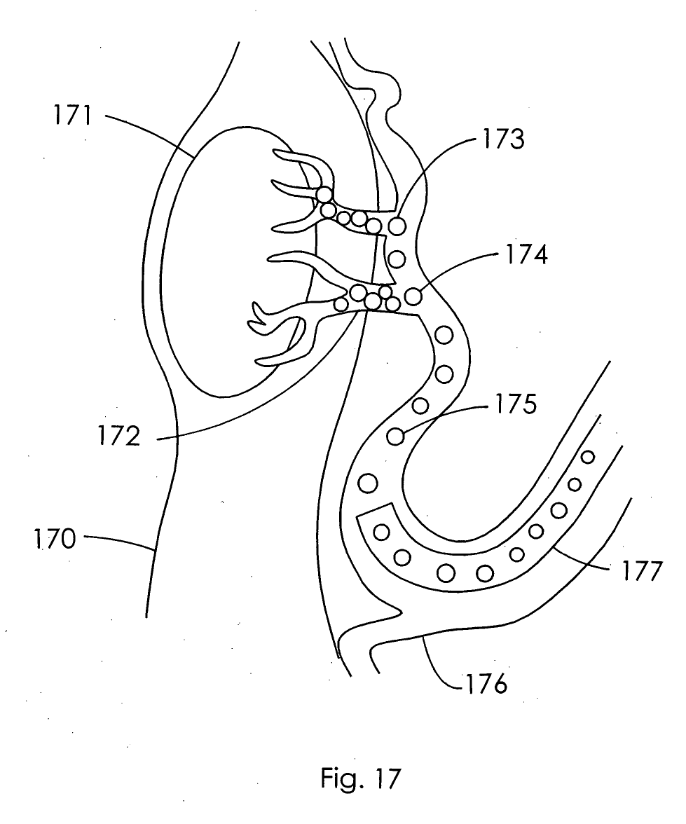

FIG. 17 is a magnified view showing the uterine with the fibroid and delivery of the particles through the catheter and the particles as they infarct the local tissues surrounding the fibroid. FIG. 17 illustrates the delivery of microparticles to a human uterus 170 through the use of a catheter system. FIG. 17 depicts the treatment of a fibroid tumor 171 fed by a fibroid tumor blood vessel 172. Microparticles 173, 174 and 175 are delivered through the uterine artery 176 by catheter 177.

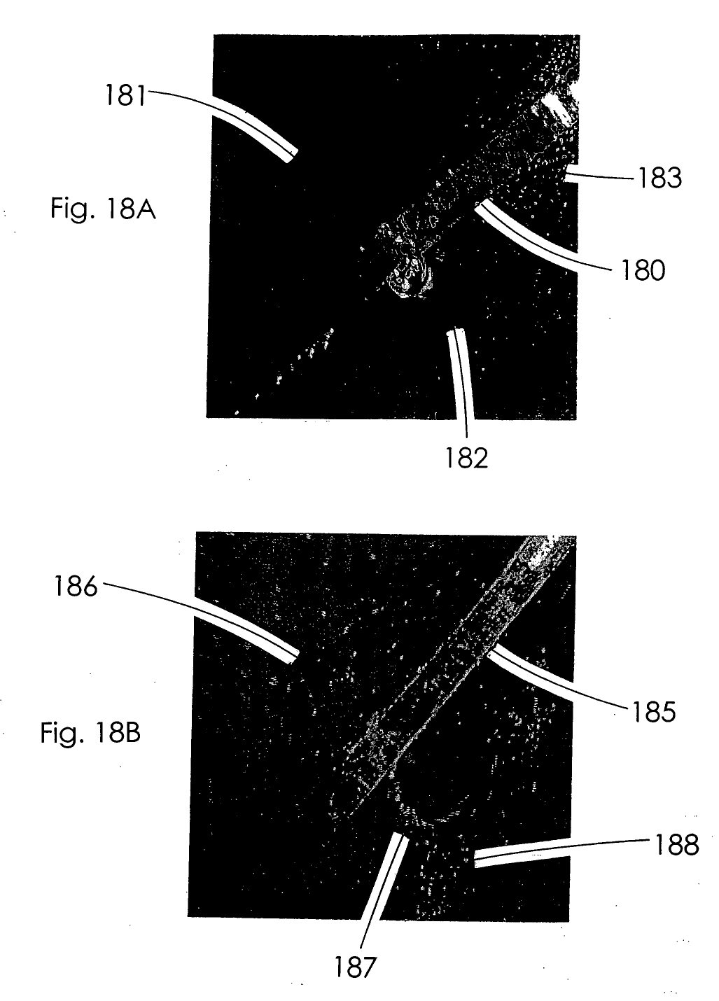

FIGS. 18A and 18B depict a bolus of microparticles with two different external diameters (10 μm and 80 μm, 19A and 19B, respectively) suspended in saline being injected in vitro through a 1.4Fr micro-infusion catheter with infusion side holes of approximately 100-150 microns (e.g., NeuroVasX™ Sub-micro Infusion Catheter, Model 100-DG-015). FIG. 18A is an image of a micro-infusion catheter with infusion side holes of 100-150 microns 180 in a saline solution 181. FIG. 18A shows injection stream 182 delivering 10 μm microparticles, e.g. microparticle 183. FIG. 18B is an image of a micro-infusion catheter with infusion side holes of 100-150 microns 185 in a saline solution 186. FIG. 18B shows microparticle injection stream 187 delivering 80 μm microparticles, e.g. microparticle 188. Both particle sizes can be delivered through the microcatheter with minimal effort.

Microparticles in some embodiments are prepared to locally deliver drugs. Further, in some embodiments microparticles can be engineered to release a substance at a controlled rate. The microparticles of the present invention can incorporate or carry one or more bioactive agents that can be locally released from the microparticle. The microparticles can act as a substrate for the controlled, sustained delivery of one or many bioactive agents (e.g., Lidocaine). In some embodiments the microparticle can provide drug delivery doses which range from nanograms to milligrams of drug per day and can be localized to the tissue in and immediately surrounding the target site. The drug-release can be sustained or can vary over time. This drug delivery is referred to as the “elution” of bioactive agents. The surface area of a microparticle also affects resorption rates and/or drug elution profiles.

Bioactive agents and additives include all compounds, solutions, materials, pure substances and mixtures of substances which may be incorporated in, carried by, impregnated in, or used in conjunction with the microparticles of the present invention. Bioactive agents can be incorporated into the microparticle as part of the manufacturing process, or at the time of clinical use. Examples of the plethora of bioactive agents includes without limitation, separately or in combination, are described herein.

Bioactive agents can include: bio-active pharmaceuticals—intended to elicit a desirable biological response. These include, but are not limited to, for example:

Gene therapies including the delivery of any gene or group of genes that code for cytokines, antigens, deficient genes, tumor suppressor, suicide, marker, receptor or any therapeutic gene (i.e. VEGF or FGF), through any gene delivery vector such as retroviruses, adenoviruses, adeno associated viruses, herpes simplex viruses, POX virus, plasmid DNA, naked DNA, and RNA transfer;

Chemo-toxins to locally treat cancerous tissues Antineoplastics (e.g., doxorubicin, cisplatin, mitomycin, actinomycin, paclitaxel, etc.);

Alkylating agents (e.g., carboplatin, and/or melphalan);

Antibiotics (e.g., daunorubicin, mithracin);

Antimetabolites (e.g., methotrexate, bisphosphonate);

Hormonal agonists/antagonists (e.g., nilutamide);

Anesthetic agents to facilitate pain management (e.g., lidocaine, bupivacaine, dibucaine, xylocaine, ropivacaine, nesacaine, mepivacaine, etidocaine, tetracaine, or mixtures thereof);

Radio-isotopes which provide localized radiation therapy (e.g., iodine-131, strontium-89, samarium-153, iridium-192, boron-10, lutetium-177, phosphorus-32, actinium-225, yttrium 90);

Energy absorbing materials which can concentrate externally applied energy (e.g., microwaves) to achieve a therapeutic effect (e.g., treat hyperthermia);

Colorants to differentiate microsphere type, e.g., FD&C Blue No. 1 (Brilliant Blue FCF), FD&C Red No. 2 (erythrosine) and FD&C No. 5 (tartrazine);

Antimicrobial substance (e.g., silver, chlorohexadine, triclosane);

Magnetic agents, which alter the behavior of microparticles in magnetic fields, magnetic resonance imaging iteration (e.g., ferrous metals); and

Agents directed at enhancing visibility (e.g. gold, tantalum) by diagnostic imaging modalities (e.g. radiographic, ultrasonic)

Microparticles are capable of the sustained elution of a bioactive agent over a period ranging from hours to months.

Other bioactive agents, additives and substance which may be incorporated into microparticles include, but are not limited to, organic and inorganic compounds and molecules, amino acids, proteins, enzymes, nucleic acid bases, bacteria, viruses, antibiotics, antibodies, antigens, prions, fats, nutrients, vitamins, elements and mixtures thereof.

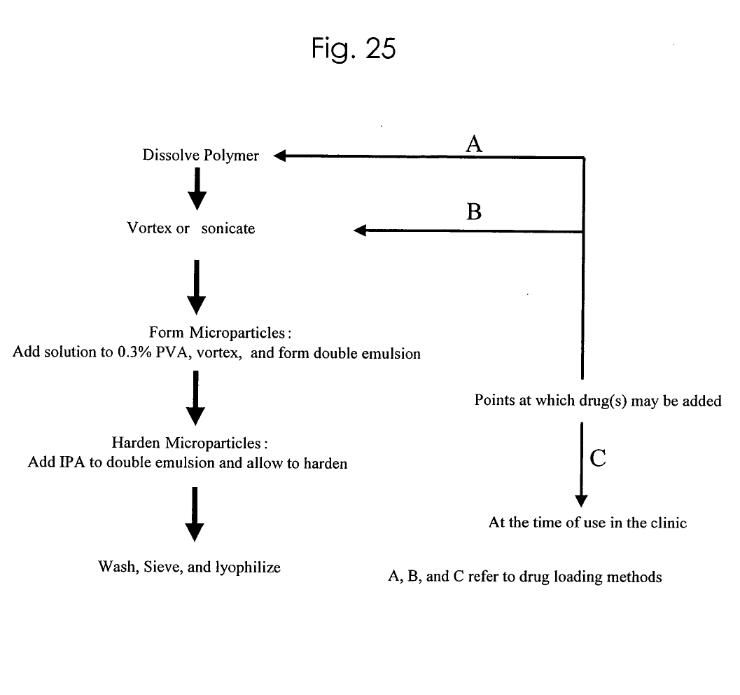

Microparticles can be adapted for the timed or time release of bioactive agents or additives. FIG. 25 is a flow diagram showing an overview of fabrication methodologies used to create particles of the present invention. The branch points, A, B and C, refer to different positions at which drugs, bioactive agents, or additives, and their mixtures may be loaded into the present invention. Bioactive agents, drugs, or additives and their mixtures can be incorporated directly into the base polymer in a soluble or insoluble form, incorporated into the void spaces in a soluble or insoluble form (19C), or adsorbed onto or adsorbed into, the microsphere.

Other typical points at which bioactive agents and additives are added include after washing, in a storage solution, in a transport solution, in a carrier solution, or any time where the microparticle or its components are brought into contact or mixed with, any bioactive agent or additive.

Additionally, microparticles of the present invention may be configured to encapsulate biosensors, diagnostic devices, or microtherapeutic machines (e.g., nanobots).

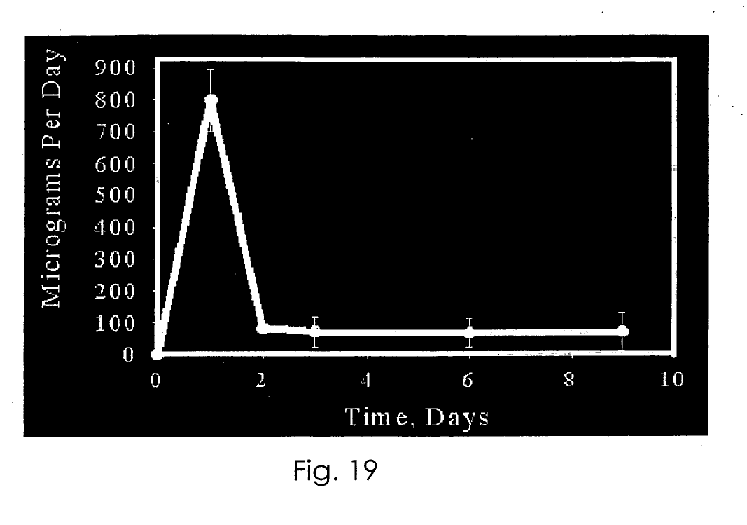

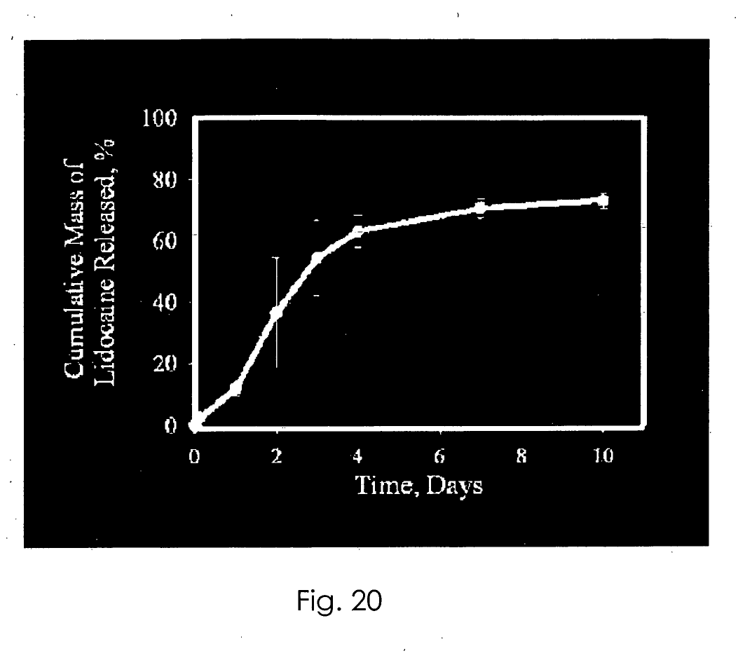

FIG. 19 is a graph of the controlled release of lidocaine from one embodiment of microparticles. See example 9 below. FIG. 12A and 12B depict a surface SEM of high lidocaine dosed particles showing a smooth spherical configuration. FIG. 20 presents the normalized cumulative mass of lidocaine released from the prototype high lidocaine dose particles at 37° C. in phosphate buffer solution (PBS). See Example 11 below.

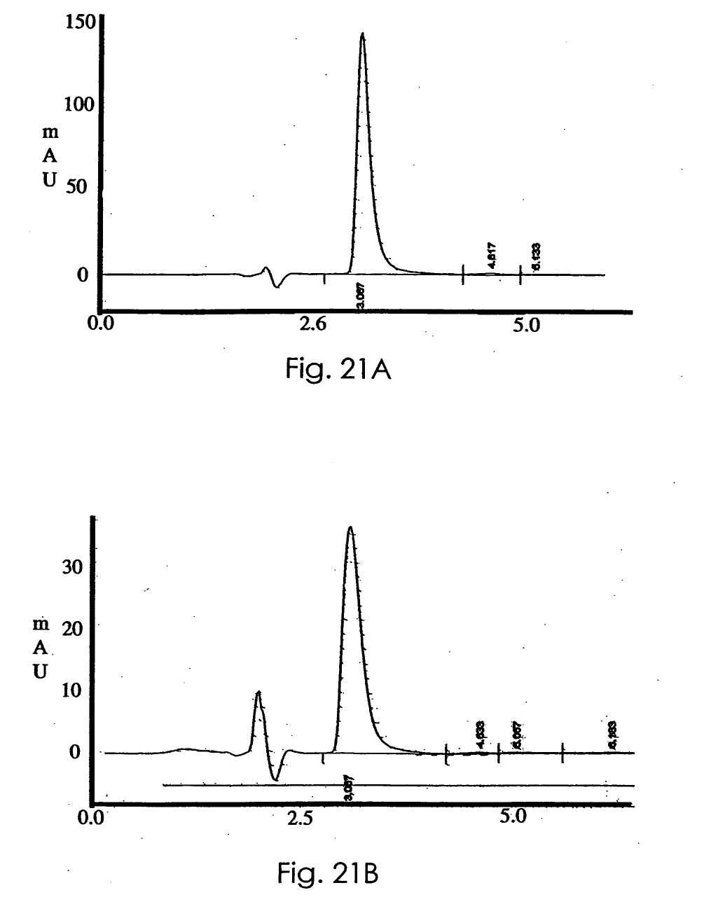

FIGS. 21A and 21B provide an example of an embodiment having lidocaine stability as demonstrated by high performance liquid chromotography (HPLC) testing. The FIG. 21A depicts HPLC test results of lidocaine eluted from the microparticles. Stability is evidenced by the common peak at 3.1 min. and the similarity of peak shapes. See Example 10 below.

Ischemic pain at the site of origin can be accomplished by blocking nerve signals in and immediately around the target site through the delivery of drug agents by the microparticles.

Visualization agents can also be delivered by the inventive microparticles. Examples of visualization agents which may be utilized with the invention include, but are not limited to, colorants or dyes. In one embodiment, the bolus of microparticles includes a visualization agent having a substance that is visible under fluoroscopy. Fluoroscopically visible substances which may be used with the present invention include, but are not limited to, gold particles.

Microparticles have a “life cycle,” “resorption profile,” or “degradation profile”. After injection into a patient, as time progresses, hydrolytic and/or enzymatic degradation or decomposition occur. Typically, no physiologically significant amount of a given microparticle remains after a period of time (typically greater than 30 days). After 270 days complete resorption of microparticles is typical.

Microparticle life cycles, or degradation rates, can vary broadly. Microparticle lifetimes in the patient can range from days to months to years. The microparticles of the present invention can be adapted to persist for greater than 30 days within the vasculature.

The microparticles of the present invention in some embodiments are fully resorbed more than 30 days after the embolotherapeutic effects are achieved. Resorption is complete in some embodiments is from 30-180 days. Shorter time periods for resorption on the order of hours to days can be employed (e.g., 6 hrs, 12 hrs, 1 day, or 15 days). After resorption is complete, no physiologically significant embolic agent is left behind within the patient that might, at some future date, migrate into adjacent vasculatures beyond the original target site and cause unwarranted embolization of healthy tissue. In some embodiments, no permanent residue of the embolic agent remains. If desired, the microparticles can be designed to persist indefinitely.

Microparticles typically exhibit a resorption rate that exceeds the duration required to achieve the clinical objectives of the embolization procedure (e.g., 1-6 months).

The time to the completion of resorption of a microparticle is dependent on the choice of base polymer chemical composition. In PLGA embodiments, one compositional variable that affects the resorption rate is the percentage of the polymer, which may be lactic acid as compared to the percentage of glycolic copolymer. The ratio of lactic acid to glycolic acid is selected in accordance with the desired resorption characteristics as discussed herein. The molar composition of lactic acid to glycolic acid can range from 0 mol % to almost 100 mol %. Thus, the ratio of lactic acid to glycolic acid can range from 0 to almost 1.0. In some embodiments, a microparticle ratio of lactic acid to glycolic acid can be from 0.25 to 0.75. The effect on resorption is to adapt resorption time typically from short time periods, e.g., minutes, hours, up to 270 days or greater. Lactic or glycolic acids have a longer degradation time as compared to copolymer ratios. As the composition approaches 50 mol % lactic acid, 50 mol % glycolic acid (i.e., a ratio of 1:1) the degradation time is further reduced for a given molecular weight. Higher molecular weight polymers take longer to degrade. High molecular weight polylactic acid can take on the order of years, while low molecular weight can be degraded within time periods greater than 1 week.

Total void volume and void distribution within the microparticle can affect the hydration of the microparticle. Total microparticle volume and void or fissure distribution within the microparticle can affect hydration. Typically, as total volume becomes larger and the distribution of voids become more dense and more interconnected the hydration rate increases. In an embodiment having a small total volume and few distributed voids the hydration rate is low (e.g., a period of 1 or more weeks). This can result because as the water diffuses through more solid polymer.

The ester bonds of the polymer backbone are broken through hydrolysis, resulting in a continual decrease in the polymer molecular weight until the individual polymer components such as lactic and glycolic acid are produced and solubilized.

In the microparticle, as the polymer continues to degrade, a loss in mechanical strength occurs. In one embodiment, a point is reached at a time greater than 30 days, where degradation results in a microparticle that is no longer compression resistant. Compression resistance in one embodiment was lost when the average polymer molecular weight was reduced by more than 15% and the microparticles had undergone a gross mass loss of 10% or more of polymer water-soluble chains.

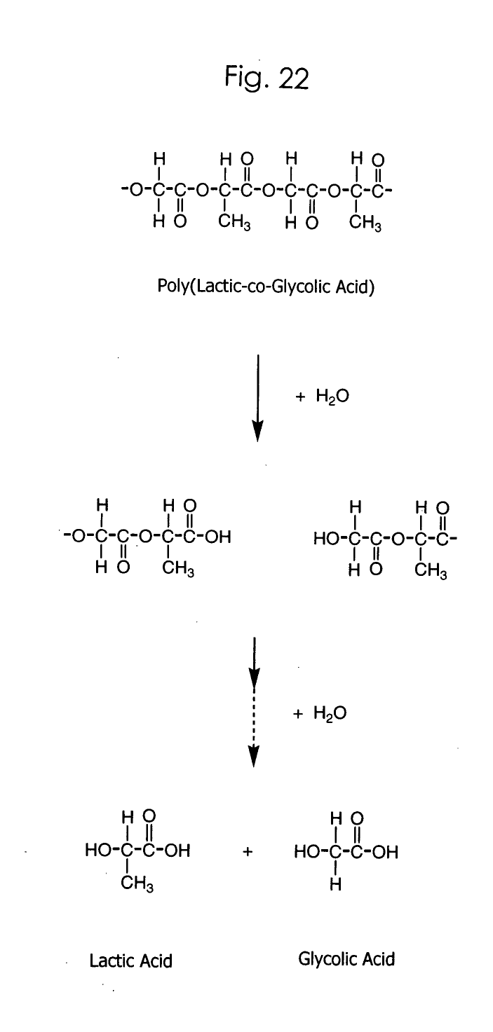

In one embodiment of microparticles of the present invention, degradation occurs through the random hydrolysis of the backbone of the base polymer (e.g., PLGA), and to a lesser extent enzymatic degradation in vivo. Degradation products (lactic acid and glycolic acid per FIG. 22) are eliminated from the body either through metabolic pathways or by direct renal excretion. In a PLGA base polymer embodiment the degradation rate can continue to increase in a nonlinear fashion as the copolymers of PLGA approach an equimolar ratio.

Microspheres in some embodiments of the present invention are fabricated from one or more compression resistant, biocompatible materials which allows for controlled bioresorption (i.e., “biodegradation,” “resorption,” “bioabsorption,” or “absorption”).

In one embodiment, resorption of the microparticles of the present invention occurs because the polymer chains have become soluble and are removed from the embolization site and body. Resorption can occur in as short a period as about 30 days for low molecular weight 50 mol %:50 mol % d,I-PLGA (d form:I form, ratio of enantiomers) formulations of 10,000, or more than a year higher molecular weight PLA weight PLA of approximately 150,000 or more.

When microparticles are embolized within a tissue bed which is no longer perfused by significant quantities of blood, the rate of hydrolysis can become autocatalytic as the removal of lactic and/or glycolic acid byproducts is curtailed and the local environment becomes acidic.

Additionally, since the hydrolytic process degrades the entire particle, no biologically significant residual foreign body is retained within the device recipient. The material which constitutes the microparticle is eventually removed from the body and the mass retention is at a level not biologically significant and even as low as to be undetectable.

As microparticles hydrolyze, they typically elicit an endoluminal fibroplastic response from the host vessel. The fibroplastic responses are generally initiated within 1-21 days of lodgment. The fibroplastic response can provide a durable tissue obstruction that is not amenable, or which prevents, recanalization of the original vessel. In some applications, the new tissue is fibrotic in nature.

Some embodiments of microparticles of the present invention elicit a characteristic biological response of inflammation.

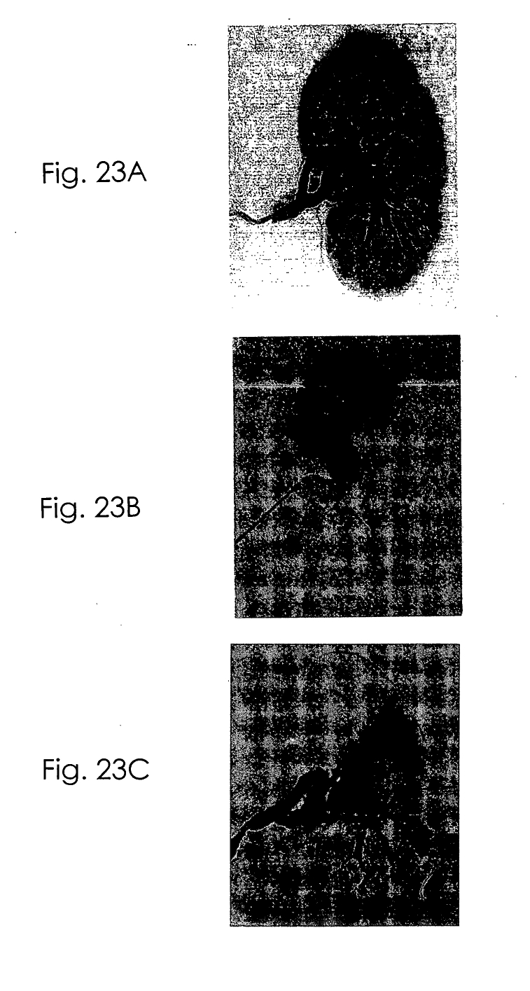

The sequence of angiographic images in FIGS. 23A-C provides an in-vivo demonstration on the selectively catheterization and acute embolization of the canine kidney. See Example 6 below.

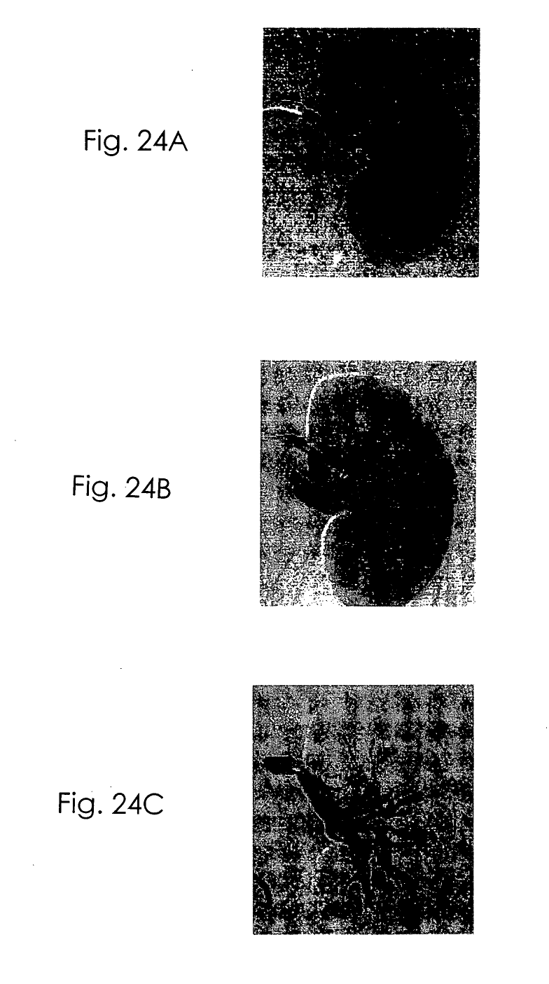

FIGS. 24A-C depicts a sequential embolization procedure of a canine kidney conducted with microparticles of two different sizes. See Example 7 below.

FIG. 22 shows the degradation of PLGA and poly(alpha-hydroxy esters) in general. The ester bonds (carbon oxygen carbon bonds) of the polymer backbone are broken through hydrolysis, resulting in a continual decrease in the polymer molecular weight until the individual polymer components such as lactic and glycolic acid are produced and solubilized.

The biodegradation of and tissue reaction to poly(α-hydroxy ester) microspheres has been studied by evaluating intramuscular injections of a copolymer of 50 mol %:50 mol % poly(DL-lactide-co-glycolide) (d form:I form, ratio of enantiomers) microcapsules (mean external diameter=30 μm) in rats using dissecting and conventional light microscopy, as well as scanning electron microscopy (SEM) and transmission electron microscopy (TEM). Post-implantation, a minimal localized acute myositis was seen initially at the injection sites. By day 4, a few small foreign body giant cells were present participating in the minimal foreign body response. Later, the inflammatory cells decreased and the individual microcapsules were walled off by immature fibrous connective tissue and large syncytial foreign body giant cells. By Day 35, definitive changes in some microcapsules, consisting of a granular and slightly eroded appearance of the internal matrix, were seen by SEM. By Day 42, the outer rims of the microcapsules were extensively eroded. At Day 56, the inflammatory and connective tissue reactions were almost completely resolved and biodegradation continued so that only remnant pieces of the microcapsules were present at Day 63. Phagocytosis did not seem to be an important factor in the biodegradation processes.

There is a known benign bioresponse for poly(α-hydroxy ester)based microspheres. The rate of poly(α-hydroxy ester) microsphere degradation increases in proportion to the glycolic unit content in the lactic chains. In-vivo degradation in the hepato-portal circulation of a rat model ranged from approximately 6 to 12 weeks for microsphere formulations that included from 75 mol %:25 mol % to 90 mol %:10 mol % lactide to glycolide ratios, respectively.

The degradation time (i.e., 6-12 weeks) of known poly(α-hydroxy ester) microspheres is consistent with the clinical objectives and timeframes of preoperative embolization procedures. If post-embolization neurosurgical procedures are indicated, they are most frequently performed in the first week following the embolization procedure. Frequently, embolization is performed immediately prior to surgery. The maximum time from embolization to surgery appears to be on the order of 72-76 days. Consequently, the poly(α-hydroxy esters) microspheres, despite biodegradation, are durable enough to achieve preoperative embolization in cases where surgery is planned.

Biodegradation rate can be a function of molecular weight in one embodiment where the base polymer has an average molecular weight of 10,000 the biodegradation time in approximately 30 days or less. In another embodiment where the average molecular weight is 150,000 biodegradation did not occur until greater than one year had passed.

The durability of vascular occlusion achieved with poly(α-hydroxy ester) microspheres is augmented by the biological response they ellicit. It is know that after embolization of rat livers, histological analyses shows that during microsphere degradation, the inflammatory response could be characterized as a moderate foreign-body reaction. The inflammation process was observed to occur in three steps, independent of polymer formulation. First, sub-acute inflammation, during which macrophages, lymphocytes, and occasionally foreign body giant cells surround the microsphere. The second step is characterized by an increase of the inflammatory reaction, while the microspheres become misshapen and the embolized region infiltrated by foreign body giant cells, lymphocytes and fibroblasts. In the third step, inflammation was observed to decrease. When degradation of the microspheres is complete, no remnants of inflammation were observed nor was purulent inflammation or hemorrhage.

The manufacture of microparticles of the present invention typically involves the steps as set forth below. FIG. 25 illustrates typical manufacturing steps for microparticles.

One or more base polymers are selected. Generally, base polymers to be considered for inventive microparticles are PLA, PGA, PLGA, PLA-TMC, PGA-TMC, PLGA-TMC, or other bioresorbable base polymers. One or more base polymers are then dissolved in solution forming a base polymer solution. The base polymer solution can be an organic solution, an aqueous solution, or a multi phase solution. The nature of the solution can be varied in view of the selection of solvent or solvents and the choice of one or more base polymers.

A polymer based solution is then added to an aqueous or organic internal phase solution. The solution to which the dissolved polymer is added is referred to as the internal phase solution. This internal phase solution can be either an aqueous phase or an organic phase as long as it is distinguishable from the properties of the polymer base solution. The internal phase solution typically becomes encapsulated, or contained within the microparticle upon its formation. It should be understood that the inventive microparticles can be created by not only adding the polymer base solution to the internal phase solution, but in some embodiments the solution serving as the internal phase solution can instead be added to the polymer base solution. In embodiments where the internal phase solution is added to the polymer base solution, the internal phase solution may be added in copious amounts in excess of the polymer base solution, or it can be added in amounts sufficient to allow formation of microparticles.

Once the polymer base solution is added to, or brought together with, the internal phase solution, the combined mixture is blended. This combined solution is referred to as the microparticle base solution. This solution may be vigorously mixed by vortexing, shaking, blending, sonication, or any other mean which applies shear forces and mixing forces to the microparticle base solution. This action of shearing and mixing is referred to as the microparticle origination step.

The external aqueous phase to which the polymer organic solution is added to form microparticles contains polyvinyl alcohol (for our examples 0.3 wt %). PVA acts as an emulsifier and prevents the unhardened microparticles from fusing together. Also, in an optional hardening step the manufacturing technique can employ the addition of isopropyl alcohol (IPA) to extract the organic from the polymer organic solution and cause the polymer to precipitate. This results in near complete hardening of the microparticles within an hour. It is also possible to not add the IPA and allow the microparticle to harden over time, e.g., greater than 1 hour to 1-10 days, as the organic phase moves from the polymer organic particle to the aqueous phase then into air.

During the manufacture of the microparticles, an optional coating step may be employed. The coating may be sprayed onto a finished microparticle, or alternatively applied by immersion into a solution containing the substances to be deposited as a coating. Optionally, the microparticles may be washed to remove excess coating.

Once the desired microparticles have been manufactured, they are typically washed, sieved and lyophilized.

The washing of the microparticles involves entrapping the microparticles on a seive of the smallest size selection, and running water over the microparticles for 1 to 2 minutes. Alternately, the microparticles may be collected in a centrifuge tube, quickly spun down at a rate of revolutions per minute (rpm) of not more than 1200, decanted and fresh water added. This may be repeated as necessary.

The sieving of the microparticles involves pouring the manufactured microparticles over a stack of sieves that contains the largest screen size on top and the smallest screen size on the bottom. The desired size range can, for example, be collected on top of the bottom screen. Lyophilization includes freezing of the microparticle before placement onto the lyophilizer.

The inventive microparticles are capable of carrying a broad variety of bioactive agents and additives. Bioactive agents and additives may optionally be within the originally selected base polymer, or polymers, as well as optionally in the internal phase solution. Bioactive agents and additives may optionally be provided by dissolution in the solvent in which the base polymers are dissolved, added to the base polymer solution, added to the internal phase solution, provided to the solution being mixed in the microparticle phase added during hardening, or coating, or added during the washing phase. Further, bioactive agents and additives may optionally be added, absorbed or adsorbed prior to use (e.g., injection) or at the time of use of the microparticles.

An overview of a summary of a fabrication process which may be employed in the manufacture microparticles of the present invention is provided in the accompanying flowchart of FIG. 25. For PLGA base polymer microparticles, a known mass of bioresorbable base polymer (i.e., PLGA, Alkermes, Inc.) is dissolved in an organic solvent, chloroform (i.e., CHCl₃, Sigma, Inc.), and thoroughly mixed by vortexing the solution. Other organic solvents such as ethyl acetate (i.e. CH₃COOCH₂CH₃, Sigma, Inc.) or methylene chloride (i.e. CH₂ C1₂, Sigma Inc.) are also commonly used. A prescribed quantity of water for the internal phase is added to the solution. This quantity has a total volume of less than the total polymer and organic solution volume. Vortexing and/or sonocation incorporates the internal aqueous phase. Approximately 4 ml of this solution is transferred to a test tube, containing approximately 15 to 20 ml of 0.3 aqueous PVA (Fisher Scientific International, Inc.), vortexed and poured into a 300 ml beaker containing 150 ml of 0.3 wt % aqueous PVA. This emulsification process is repeated as necessary.

PVA is used as a surfactant to prevent microparticle aggregation. The resulting emulsification is vigorously mixed using a magnetic bar. This re-emulsification process forms shear-induced spherical microparticles comprised of bioresorbable base polymer to which 100 ml of 2 vol % aqueous isopropanol (IPA; Fisher Scientific International, Inc.) is subsequently added. Hardening of the microparticles results as extraction of the dichloromethane to the external alcoholic phase precipitates the dissolved base polymer. The system is stirred for a sufficient period of time (i.e., from 1.5 to 2 hours) to assure adequate extraction of the solvent. Finally, the formed microparticles are sieved to prescribed size ranges, rinsed in water, and lyophilized to a produce fine powder. Packaging and sterilization can then be performed.

As previously indicated, bioactive agents or additives can be added at various stages of the microparticle fabrication process. Bioactive agents can be considered as a subset of additives. As examples, bioactive agents, drugs, additives and their combinations can be incorporated as indicated in FIG. 25 at least at points A, B, and C. The bioactive agent or additive optionally can be added at point not limited to:

The polymer organic solution (bioactive agent(s) may be soluble or insoluble in organic);

The internal aqueous phase (bioactive agents(s) can be soluble or insoluble in aqueous phase);

After the final steps of manufacturing (wash, sieve, and lyophilize) when the particles are in a powder or dry form, by:

Physical mixing with a bioactive agent (e.g., drug) or additive. For example, before injection of microparticles in a patient, a liquid can be added in which a bioactive agent is fully or partially dissolved;

Physical mixing with a drug solution that may or may not be combined with additional liquids for injection;

Spray coated with a drug; or

Physical mixing of other drug containing materials such as other particles with a drug.

Incorporation into or on a coating layer,

Adsorbed or absorbed onto or into a microparticle at a time of use.

Density manipulation of a microparticle can be accomplished during manufacturing through a double emulsion technique in which an internal aqueous phase is incorporated into the organic polymer solution before the formation of the primary particle. Solid, low-density material (e.g., gelatin, PLGA-PEG, PVA, collagen, chitosan, chitin, albumin, alginate, polyethylene oxide, polyvinyl alcohols, pectin, amylose and fibrinogen) may be incorporated instead of the internal aqueous phase.

The microspheres of the present invention allow the incorporation of multiple bioactive agents and/or multiple bioresorbable polymers (e.g., bio-polymers of the same family having different degradation rates).

There are multiple methods of drug formulation with the base polymers for sustained controlled release for 1 to more than 45 days of one or more pharmaceutical agents.

FIG. 25 is a flow diagram showing an overview of the fabrication methodologies used to create particles of the present invention. The branch points, A, B, and C refer to different positions at which drugs, bioactive agents, or additives, may loaded into the present invention. Bioactive agents can be incorporated directly into the base polymer in a soluble or insoluble form, incorporated into the void spaces in a soluble or insoluble form (19C), or adsorbed onto, or absorbed into, the microsphere.

Other typical points at which bioactive agents and additives are added include after washing, in a storage solution, in a transport solution, in a carrier solution, or any time where the microparticle or it components are brought into contact, or mixed with, any bioactive agent or additive.

Treatment with microparticles of the present invention typically includes, but is not limited to, embolization or delivery of microparticles having one or more bioactive agents or additives.

The microparticles can be used without limitation in the embolization of malignant or benign tissue masses often occurring in the brain, liver, uterus, ovaries, spine, head, neck, breast and to a lesser extent in other locations. The microparticles can be delivered by injection. The same procedural techniques utilizing interventional radiology techniques involving catheters, angiography, and syringes can be employed with the inventive microparticles.

The microparticles of the present invention are designed to create a blockage upon lodgment or accumulation of microparticles forming an occlusion upon injection and reaching a target location. A blockage can result in some embodiments as size-matched microparticles lodge in a target blood vessel inhibiting perfusion through the target vessel. The microparticles lodge within target vessels that have become distended and loaded with microparticles obstructing blood flow when injected into a target vasculature.

Blockages can be achieved from as little as one microparticle or several or many microparticles can constitute the blockage.

Microparticles of this invention may also be utilized in a manner that does not require embolization of a conduit as a procedural objective. For example, microspheres of the present invention may, without limitation, be directly injected into peri-luminal tissues for tissue bulking applications, directly into tissue masses for cancer or myocardial treatments, into the wall of a blood vessel or biological conduit to treat intramural disease, or injected into the bloodstream to achieve a bioactive benefit. The microspheres of the present invention are amenable to these and similar non-embolizing applications.

Embolic particulates are typically delivered to selected embolization sites via transcatheter injection. Delivery catheters having a configuration (e.g., external diameter, length, shape) appropriate to the vascular target and size of the embolic agent are appropriate. The inventive microparticles can be delivered through infusion catheters of varying internal diameters, including microcatheters. For typical embodiments the internal diameters of catheters utilized with the present invention are from about 150 microns to about 2 mm catheters. However, catheters with significantly smaller internal diameters (e.g., 50 microns) or larger (e.g., 5 mm) may be employed as necessary for delivering nanoparticles or large external diameter particles respectively. Embolic agents can be delivered through small-bore 100 micron to 1000 micron internal diameter infusion catheters. Injection through microcatheters allows the microparticles to be delivered to tumor sites and facilitates targeted therapy. The smaller the microparticle the smaller the vessels are which can be embolized. One embodiment targets small tumors. Smaller catheters may minimize vessel spasm and improve embolization procedure success rate.

Fluoroscopic visualization of catheter delivery and the injection procedure ensures accurate placement of embolization devices can be achieved in some embodiments. The microparticles may include, or be mixed with, a radiopaque contrast agent prior to injection. As with conventional embolization procedures, the ratio of injection medium to embolic particulates is dependent on the clinical objectives of the embolization procedure.

In one embodiment of the present invention, a delivery system for microparticle utilization includes the following a bolus of microparticles having optional bioactive agents or additives, delivery apparatus adapted to contain, or containing, the bolus of microparticles. Further the delivery apparatus is configured to inject the bolus microparticles and a carrier solution into a patient.

The microparticles of the present invention may be supplied in a powder-like form or suspended in a transport solution, carrier solution, or injectate. Some embodiments can be supplied in a single use, sterile vial containing a pre-measured amount of microparticles (FIG. 14). Alternatively, the microparticles may be pre-packaged in a kit-type system that can include, but is not limited to, the microparticles which can optionally have bioactive agents or additives, a pre-measured portion of injection solution (e.g., radiopaque contrast agent and saline whose density is optimized for use with the microparticles), a means of mixing the microspheres and the injection solution (e.g., saline solution, carrier solution, or contrast agent solution) and a means to facilitate injection of the suspension through a catheter. Commercially available mixing/injection systems that might fulfill the requirements outlined above with minimal modification include the Becton Dickenson MONOVIAL™ and the Vetter LyoJect™ syringe, both of which can be used to reconstitute dry pharmaceuticals prior to injection.

Sterilization of the microparticles can be achieved by one of any number of validated, non-hydrous methods including, but not limited to: radiation, ultra violet light, or ethylene oxide.

Without intending to limit the present invention, the following examples specify how the present invention can be made and tested.

### EXAMPLES

Microparticles of the embodiments in Examples 1, 2, 3, 7, 16 and 18, were manufactured by a modification of a double-emulsion-solvent-extraction technique. This process enabled the fabrication of microparticles having high density, low density, and microparticles that incorporated a bioactive agent (e.g., lidocaine) which was dissolved directly into the bioresorbable polymer.

### Example 1

### High Density Microparticle Fabrication

Microparticles of the present invention having a relatively small volume of voids were fabricated using the following process:

1) A 75 wt/vol % (weight/volume %) solution of PLGA (85:15 copolymer mole ratio) with chloroform was prepared.

2) 2 mL of 75 wt/vol % PLGA was placed in a 20 mL screw top test tube and warmed under tap water to lower the viscosity.

3) 0.5 mL of de-ionized (DI) water was added to the 2 mL of 75 wt/vol % PLGA.

4) An emulsification was created by vortexing the mixture for 1 minute on setting #8 while maintaining the tube perpendicular to the vortex and holding the test tube at the apex.

5) The emulsion was then rapidly poured into a 50 mL test tube containing 10 mL of 0.3wt/vol % PVA.

6) A double emulsion was formed by vortexing the PLGA/Water/PVA emulsion for 1 minute on setting #8 while maintaining the tube perpendicular to the vortex and holding the test tube at the apex.

7) The PLGA microparticles were then rapidly poured into a 500 mL beaker containing 250 mL of 0.3 wt/vol % PVA while stirring.

8) 250 mL of 3.0 wt/vol % IPA (1:1) was then added to the beaker containing the PLGA microparticles.

9) The PLGA microparticles were allowed to harden for 2 hrs.

10) The microparticles were sieved using USA Standard Testing Sieves (ASTME—11 Spec.). Microparticles of 90-180 μm were collected.

11) The microparticles were washed in the sieve with copious amounts of DI water.

12) The microparticles were then transferred to a screw cap plastic vial.

13) The microparticles were immediately frozen at −80° C.

14) The microparticles were lyophilized overnight (approximately 12 hours).

### Example 2

### Low Density Microparticles Fabrication

Microparticles of the present invention having a relatively large void volumes were fabricated using the following process:

1) A 25 wt/vol % solution of PLGA (85:15 copolymer mole ratio) with chloroform was prepared.

2) 6 mL of 25 wt/vol % PLGA in a 20 mL screw top test tube was warmed under tap water to lower the viscosity.

3) 2.0 mL of DI water was added to the 6 mL of 25 wt/vol % PLGA.

4) Follow Steps 4-14 as above.

### Example 3

### Variable Void Volume/Variable Density

An experiment was conducted to evaluate the effects of process variables on resulting microparticle configuration (spherical vs. non-spherical) and microparticle density relative to de-ionized (DI) water. Process variables which were examined in this experiment included:

1) wt/vol % of PLGA to CHCL₃ (25 wt % to 75wt %);

2) aqueous phase additive (0.5 ml to 2.0 ml); and

3) adjunctive sonication during the initial emulsification step.

The methods used in these experiments were identical to those described above, with the following modifications:

1) an 85 wt/vol % PLGA base polymer was used instead of 75 wt/vol %,

2) the inclusion of lidocaine loading of the PGLA polymer (approximately 50% by weight) in to the dissolved base polymer at step A in the flowchart depicted in FIG. 25, and

3) the addition of the sonication step in a sub-group of samples. Results of this experiment are presented in the following tables (i.e., Tables A-D):

TABLE A
Microparticle Configuration
Variable Void Volume Experiment - Without Sonication

| | wt/vol % PLGA in CHCL₃ | | | | | |
|---|---|---|---|---|---|---|
| | (Using constant weight of 1.5 g of 85:15 PLGA and CHCL₃ as the Oil | | | | | |
| | Phase) | | | | | |
| Aqueous | 75% | 65% | 55% | 45% | 35% | 25% |
| Phase H₂O (mL) | (1.5:2.0) | (1.5:2.3) | (1.5:2.7) | (1.5:3.3) | (1.5:4.3) | (1.5:6.0) |
| 0.5 | Sphere | Sphere | Sphere | Sphere | Sphere | Sphere |
| 1.0 | Sphere | Sphere | Sphere | Sphere | Sphere | Sphere |
| 1.5 | Sphere | Sphere | Sphere | Sphere | Sphere | Sphere |
| 2.0 | Sphere | Sphere | Sphere | Sphere | Sphere | Sphere |

TABLE B
Microparticle Density Relative to DI Water
Variable Void Volume Experiment Results - Without Sonication

| | wt/vol % PLGA in CHCL₃ | | | | | |
|---|---|---|---|---|---|---|
| | (Using constant weight of 1.5 g of 85:15 PLGA and CHCL₃ as the Oil | | | | | |
| | Phase) | | | | | |
| Aqueous | 75% | 65% | 55% | 45% | 35% | 25% |
| Phase H₂O (mL) | (1.5:2.0) | (1.5:2.3) | (1.5:2.7) | (1.5:3.3) | (1.5:4.3) | (1.5:6.0) |
| 0.5 | S | S | S | 50:50 | F | F |
| 1.0 | S | S | S | 50:50 | F | F |
| 1.5 | S | S | S | 50:50 | F | F |
| 2.0 | S | S | S | 50:50 | F | F |

S = Sink,
F = Floats,
Approx. Ratio = Sink:Float

TABLE C
Microparticle Configuration
Variable Void Volume Experiment - With Sonication

| | wt/vol % PLGA in CHCL₃ | | | | | | | |
|---|---|---|---|---|---|---|---|---|
| Aqueous | (Using constant weight of 1.5 g of 85:15 PLGA and CHCL₃ as the Oil Phase) | | | | | | | |
| Phase | 75% | 65% | 55% | 45% | 37.5% | 35% | 30% | 25% |
| H₂O (mL) | (1.5:2.0) | (1.5:2.3) | (1.5:2.7) | (1.5:3.3) | (1.5:4.0) | (1.5:4.3) | (1.5:5.0) | (1.5:6.0) |
| 0.5 | — | — | — | — | — | — | — | Sphere |
| 1.0 | Sphere | — | — | — | — | — | — | Sphere |
| 2.0 | — | — | — | — | Sphere | — | Sphere | — |

TABLE D
Microparticle Density Relative to DI Water
Variable Void Volume Experiment Results - With Sonication

| | wt/vol % PLGA in CHCL₃ | | | | | | | |
|---|---|---|---|---|---|---|---|---|
| Aqueous | (Using constant weight of 1.5 g of 85:15 PLGA and CHCL₃ as the Oil Phase) | | | | | | | |
| Phase | 75% | 65% | 55% | 45% | 37.5% | 35% | 30% | 25% |
| H₂O (mL) | (1.5:2.0) | (1.5:2.3) | (1.5:2.7) | (1.5:3.3) | (1.5:4.0) | (1.5:4.3) | (1.5:5.0) | (1.5:6.0) |
| 0.5 | — | — | — | — | — | — | — | 50:50 |
| 1.0 | S | — | — | — | — | — | — | 25:75 |
| 1.5 | — | — | — | — | — | — | — | — |
| 2.0 | — | — | — | — | S | — | 25:75 | — |

S = Sink,
F = Floats,
Ratio = Sink:Float

General Observations

1) Microparticles exhibited generally spherical geometry.

2) Microparticle external diameter varied in size from nanometers to millimeters.

3) Microparticles were made with observable void volumes incorporated into the microparticles (refer to FIGS. 16-18).

4) Sonication of the internal aqueous phase into the PLGA/chloroform emulsification phase created void volumes that were more finely and evenly dispersed than mechanical mixing (refer to FIGS. 17 and 18).

5) The variable void volume contributed to variable density of different microsphere configurations and, therefore, and different buoyancies when suspended in DI water.

6) Some microparticles with “neutral” buoyancy (i.e., a 50:50 ratio of suspended to sinking microparticles) were created in these experiments. With some fine tuning of the process conditions it would be possible to create a microparticles where the majority are “neutrally buoyant.” Selection methods (e.g., sieving can then be used to create uniformly buoyant microparticles.

7) Without sonication, a wt/vol % PLGA (85:15 mole ratio) in CHCL₃ of 45% produced microparticles that suspended uniformly in DI water regardless of aqueous phase volume. With sonication, a wt/vol % PLGA (85:15 mole ratio) in CHCL₃ of 25% produced microparticles that suspended uniformly in DI water at an aqueous phase of 0.5 mL.

8) Lidocaine loading of the microparticles did not appear to significantly impact the geometry, buoyancy or physical integrity of the microparticles.

9) Though only one drug loading was demonstrated in this previous experiment, multiple hydrophilic and/or hydrophobic drugs could be loaded into these microparticles. For example, hydrophilic drugs could be loaded into the internal aqueous phase and hydrophobic drugs could be loaded into the oil (chloroform) phase.

10) Some surface disruptions were observed after lyophilization. Presumably this was due to the removal of the internal aqueous phase. This may be due to the relatively slow freezing process employed in this experiment. “Flash freezing” in liquid nitrogen or acetone and dry ice might minimize this phenomena.

### Example 4

### In-Vitro Infusion of Microparticles

The inventive microspheres can be injected through a small internal diameter infusion catheter. Microparticles of the present invention can be also injected through conventional, low profile infusion catheters.

FIGS. 18A and 18B depict prototype microparticles of the present invention with two different external diameters (10 μm and 80 μm, respectively) suspended in saline being injected through a 1.4-Fr micro-infusion catheter with infusion side holes of approximately 100-150 microns (NeuroVasX™ Sub-micro Infusion Catheter, Model 100-DG-015). Both particle sizes can be delivered through the microcatheter with minimal effort.

### Example 5

### In-Vivo Selective Renal Catheterization and Embolization

The sequence of angiographic images in FIG. 23A-C provides a demonstration on the selectively catheterization and embolization of the canine kidney. The renal circulation is an excellent procedural model to demonstrate the benefits of an embolic agent. FIG. 23A is a contrast angiogram of the renal cortex. FIG. 23B depicts the selective catheterization of the cephalad pole of the left kidney, which was subsequently embolized with prototype microparticles of the present invention. FIG. 23C is a completion angiogram demonstrating the ability of the microparticles to be injected into the renal circulation and the acute efficacy of the microparticles to obliterate flow to the cephalad pole of the left kidney.

### Example 6

### In-Vivo Dual-Injection Embolization Technique

FIG. 24A-C depicts a sequential embolization procedure of a canine renal cortex conducted with microparticles of two different sizes. As above, FIG. 24A is a contrast angiograph of the renal cortex. FIG. 24B is an angiogram following embolization with microparticles of 80 micron external diameter (200 mg of microparticles in 12 ml of 50:50 saline:contrast). Here only the outermost periphery of the cortical circulation (i.e., smallest vasculature) is embolized. FIG. 24C is an angiogram following embolization with a larger particle size (240 microns; 200 mg of microparticles in 12 ml of 50:50 saline:contrast), demonstrating the ability of microparticles of the present invention to be injected into the renal circulation and to obliterate perfusion of the more proximal renal circulation.

### Example 7

### Local Drug-Delivery Examples

One or more bioactive agents may be incorporated into the microsphere. Fabrication of lidocaine eluting microspheres, representative of a drug-eluting embodiment, was conducted per the previously described methodology (specifically, lidocaine (Sigma Chemical, Inc.) was added at step A in the flowchart presented in FIG. 22). 3.6 g of PLGA (75:25 copolymer ratio) and 0.9 g of lidocaine were dissolved in 7.2 ml of chloroform to form a homogeneous solution. Aliquots of 3 ml were processed into microparticles with an internal aqueous phase of 0.150 mL DI water. On a theoretical loading basis, lidocaine represents 20 wt % of the initial formulation, while actual loading was determined to be approximately 8 wt %. The following section demonstrates typical findings associated with lidocaine loading of bioresorbable microspheres of the present invention.

### Example 8

### Controlled Release of Lidocaine (FIG. 19)

The average mass of lidocaine released per day at 37° C. in PBS solvent was measured with prototype microparticles. In this example, the microparticles were determined to contain 8 wt % lidocaine. The release profile, shown in FIG. 19, indicated that approximately 800 micrograms were eluted as a burst release within the first 24 hours. This initial burst release of lidocaine was followed by a continuous release of approximately 70 micrograms per day for the next 9 days. This profile demonstrates what is thought to be a clinically relevant dosing regimen for the UFE patient (Note, Xylocaine Instructions for Use specify 100 mg dose for general obstetrical analgesia).

### Example 9

### Lidocaine Stability as Demonstrated by HPLC Testing (FIG. 21)

High Performance Liquid Chromatography (HPLC) was performed to verify that the chemical composition (and functionality) of the lidocaine eluted from prototype microparticles was identical to a standardized lidocaine control. The standard is commercially available lidocaine dissolved in water. Chromatographs (FIGS. 21A and 21B) demonstrate that lidocaine is stable within the embolic particle matrix. FIG. 21A depicts the lidocaine standard, and FIG. 21B depicts lidocaine eluted from the microparticles. Stability is evidenced by the common peak elution time (3.1 min) and the similarity of peak shapes. These data suggest that the functionality of lidocaine eluted from prototype microparticles is maintained throughout processing.

### Example 10

### High Lidocaine Dose Microparticles (FIGS. 12 & 20)

Initial experimentation was conducted to characterize the maximum mass of lidocaine that could be loaded into prototype microparticles. FIGS. 12A and 12B depicts a surface SEM of high lidocaine dose microparticles showing a spherical configuration. FIG. 20 presents the normalized cumulative mass of lidocaine released from the prototype high lidocaine dose microparticles at 37° C. in PBS. These microparticles contain 56 wt % lidocaine, and can deliver 56 mg of lidocaine per 100 mg of microparticles. As such, these microparticles are thought to represent an upper limit to the delivery of lidocaine from embolic microparticles. The drug delivery occurs over a 4 day period without an early burst phase.

### Example 11

### Comparative Clinical Example

Uterine fibroids are non-cancerous (benign) tumors that develop in the muscular wall of the uterus. Although fibroids are not always symptomatic, their size, number and location can lead to problems for some women, including pain and heavy menstrual bleeding. Fibroids range in size from very tiny (<1 cm) to the size of a cantaloupe or larger (>20 cm). In some cases they can cause the uterus to grow to the size of a five-month pregnancy or more. Fibroids may be located in various parts of the uterus as depicted in FIG. 15.

Uterine fibroid embolization (UFE) involves guiding a catheter into the uterine artery under fluoroscopic guidance (FIG. 16). The doctor then injects an embolic agent into an artery which supplies blood to a fibroid tumor. This interrupts blood flow to the tumor and causes localized ischemia (FIG. 17). The contralateral artery is then treated according to most protocols.

Uterine fibroid embolization is usually an in-patient procedure that requires a hospital stay of one night. Pain-killing medications and drugs that control swelling typically are prescribed following the procedure to treat cramping and pain, which are the most common side effects. Fever is an occasional side effect, and is usually treated with acetaminophen. Many women resume light activities within a few days, and the majority of women are able to return to normal activities within one week.

UFE procedures result in tumor shrinkage, and symptom reduction. 78 to 94 percent of women who have the UFE procedure experience significant or total relief of heavy bleeding, pain and other symptoms. The procedure also appears to be effective for multiple fibroids. Recurrence of treated fibroids is very rare, and only about 3% of patients so far have moved on to surgical solutions due to treatment failure.

It is believed that the microparticles of the present invention may be successfully employed in this type of procedure with even more promising results.

### Example 12

### Embolization Efficacy and Compression Resistance Examples

The microspheres of the present invention are resistant to extrinsic compression due to physiological loads. The potential benefits of the incompressible nature of these microspheres, consequently, is best demonstrated by in-vivo experimentation conducted with prototypes fabricated with the processes outlined in FIG. 25. Histological examples from two in-vivo experiments, an acute study and a sub-chronic study, are presented below. The histological results from these experiments demonstrate modest deformation of the host vasculature to accommodate the presence of the microsphere, the absence of overt deformation or compression of the microspheres themselves, and a durable embolization result from the time of initial injection (acute) through 30 days (sub chronic).

### Example 13

### Acute Histological Example

FIG. 7 is a photomicrograph demonstrating the in-vivo mechanism of embolization with microspheres of the present invention. Shown therein is a longitudinal cross-section of an embolized arterial segment. Prototype microspheres can be observed to be lodged within this blood vessel interspersed with red blood cells and clot. Evident is the dimensional matching of the microparticle and the arterial lumen and the moderate deformation of the host vessel to accommodate the presence of the larger external diameter microspheres.

### Example 14

### In-Vitro Testing of Compression Resistance

FIG. 5 is a diagram demonstrating the in-vitro mechanism of testing for compression resistance. A syringe (50) filled with microparticles (52) suspended in a carrier solution forming an injectate (51) is injected into tube (53). The microparticles travel through tube (53) and are required to compress in order to travel through test channel (55). If the microparticles emerge in the effluent intact, then they are termed not to be compression resistant. Conversely, If the microparticles can not pass through test channel (55) and do not appear in the effluent, then they are termed compression resistant.

### Example 15

### Sub-Chronic Animal Study

An animal study was conducted to evaluate bioresorbable polylactic acid (PLA) microparticles of the present invention loaded with 8±3 wt % lidocaine when used as an embolic agent in the canine renal circulation. The external diameter of the microspheres used in this experiment had a size range of 150 to 250 μm. The elution curve of the test particulates is depicted in FIG. 19 four (n=4) kidneys were embolized with prototype microparticles (250 μl of microparticles suspended in 12 ml of fluid). Approximately 16 mg of lidocaine was delivered per animal.

Following approximately thirty days post-embolization, all animals were retrieved after contrast angiography. Gross and microscopic evaluations were performed to characterize the presence and extent of vascular (renal artery) thrombosis and renal infarction. The endpoint of the in-vivo phase was the histological evaluation at approximately 30 days post-operation.

Histological evaluation of the explanted specimens showed that treatment with prototype microparticles was durable and associated with renal infarction without evidence of tubular regeneration through 30 days (FIG. 8). Prototype microspheres were associated with small infarcts that were often localized to a single (cephalad) pole of the kidney. Inflammation in the test group may represent a response to the prototype microspheres themselves, the lidocaine or both components. Evident in FIG. 7 is the deformation of the tubular structures within which the compression resistant microspheres are lodged.

### Example 16

### Solution Concentration for Microparticles

Brain-like convoluted surfaces and smooth surfaces can be produced. For brain-like surfaces, 1.84 g PLA and 0.16 g of lidocaine was dissolved into 5 ml chloroform. A 3 ml aliquot of the solution was transferred to a test tube, 200 microliters of DI water was add, and the system was vortexed to produce a single emulsion. This single emulsion was used to produce microparticles that yielded the brain-like structures (FIGS. 13A and 13B). An identical solution of PLA and lidocaine was prepared. To this an addition amount of chloroform was added to significantly reduce the viscosity, and particles with internal aqueous phase of 200 miroliters of DI water were made. Upon SEM examination it was noted that the second batch of microparticles prepared with the reduced viscosity yielded a smoother surface.

### Example 17

### Lidocaine Loaded Microparticles for Chronic Animal Implants

3.5 grams PLGA 75:25 mole ratio (per Boehringer Ingelheim RG755)

0.875 grams lidocaine (Sigma Chemical)

6 ml chloroform

Mix ingredients above and allow to fully dissolve. Periodically placed into warm water bath and vortexing.

Separate in 2.5-4 ml portions. Add 150 microliters Diwater to each portion. Vortex for 20 sec to create first emulsion. Pour contents into large glass test tube containing approximately 20 ml 0.3 wt % PVA solution. Vortex 25 sec.

Pour particles into beaker with stir bar containing 0.3 wt % PVA solution (approximately 150 ml). Add approximately 150 ml IPA 3 vol % solution and allow to harden for 2.5 hours. Collect with sieves, DI rinse, freeze, and lyophilize.

### Example 18

### Brain-Like Microparticles with Lidocaine

1.84 grams PLA

0.16 g lidocaine

5 ml chloroform

Mix and allow to dissolve. To 3 ml solution add 200 microliters DI water and vortex. As before, pour into PVA solution, add IPA solution, and collect after sufficient time to harden.

### Example 19

### Compression Resistance Testing of Compressive and Compression Resistant Microspheres Using a Catheter with a 480 Micron Inner Diameter

CONTOUR SE™, compressible, polyvinyl alcohol (PVA) microspheres of 300-500 micron diameter were obtained (Catalog Number 76-122, Boston Scientific Corporation, Watertown, Mass.). Approximately 1 ml of these microspheres were suspended in a mixture of 50 vol % Phosphate Buffered Saline: 50 vol % Visipaque™ contrast medium. The mixture was made from 6 ml of phosphate buffered saline (PBS) (GIBCO, Life Technologies, Inc., Rockville, Md.) and 6 ml of VISIPAQUE™ 320 mg l/ml iodoxinole contrast medium (Amersham Health, Cork, Ireland). The mixture of compressible microspheres, PBS and contrast medium was then loaded into a 20 ml polypropylene syringe (Tyco Healthcare/Kendall, Joliet, Ill.). The 20 ml syringe was placed onto one port of a four-way stopcock (Catalog Number 91045, Mallickrodt Critical Care, Glens Falls, N.Y.) and a 3 ml. polycarbonate syringe (Merit Medical Systems Inc., South Jordan, Utah). was placed onto another port of the stopcock. Compressible microspheres were then transferred from the 20 ml syringe to the 3 ml syringe. The 20 ml syringe was then removed from the port of the four-way valve port was replaced by an angioplasty balloon inflation device (B. Braun Medical, Inc., Bethlehem, Pa.). An EXCELSIOR™ 1018 microcatheter (Boston Scientific, Fremont, Calif.) was placed on the last remaining port of the four-way valve. The EXCELSIOR™ 1018 microcatheter had a tapered section made of a rigid thermoplastic material located between the luer fitting on its proximal end and the beginning of the flexible section of the catheter. The tapered section of the catheter reduced from an inner diameter of approximately 4000 microns at the luer fitting to approximately 480 microns where the flexible section of the catheter began.

The compressible microspheres were transferred from the 3 ml syringe into the microcatheter. The angioplasty balloon inflation device was then used to force approximately 20 ml of water through the microcatheter. As the water, microspheres, PBS and contrast medium were passed through the microcatheter, less than 1 atm pressure registered on the pressure gauge that was mounted on the angioplasty balloon inflation device. The pressure gauge had a range of 0-30 atm.

Microspheres were observed being ejected from the distal end of the microcatheter into the glass beaker, and it appeared that all of the compressible microspheres had passed through the microcatheter after the approximately 20 ml of water had passed through the microcatheter.

The same procedure was followed using CONTOUR SE™, compressible, PVA microspheres of 500-700 micron diameter (Catalog Number 76-130, Boston Scientific Corporation, Watertown, Mass.). As with the 300-500 micron compressible microspheres, these 500-700 micron compressible microspheres were observed to pass through the microcatheter into the collection beaker at the distal end. However, with these larger microspheres, a back-pressure of approximately 1 atm was observed on the pressure gauge which was mounted on the angioplasty balloon inflation device as the compressible microspheres and PBS with contrast medium mixture were passed through the microcatheter.

The same procedure was then followed using compression resistant bioabsorbable microspheres of the present invention. Compression resistant, bioabsorbable microspheres of 85 mol % PLA: 15 mol % PGA were prepared using a polymer with an inherent viscosity of 0.65 dl/gm in chloroform at 30 ° C. They were sized dry, using two standard testing sieves conforming to ASTM standard specification E 11. The two sieves were stacked together with a No. 25 sieve having approximately 707 micron sized openings on top and a No. 35 sieve having approximately 500 micron sized openings on the bottom. The microparticles were placed onto the No. 25 sieve and then both sieves, while still stacked, were agitated to encourage the microparticles with diameters smaller than 707 microns to migrate through the No. 25 sieve. Microparticles with diameters smaller than 707 microns but greater than 500 microns accumulated on the surface of the screen of the sieve on the bottom of the stack, which was a No. 35 sieve with approximately 500 micron sized openings. The microparticles that had accumulated on the surface of the No. 35 sieve were then collected for the in-vitro compression resistance test. Through the process of sieving, these microparticles were determined to be in the approximately 500-707 micron size range.

Approximately 0.1 gm of the 500-707 micron sized microparticles were put into approximately 12 ml of a mixture of 50 vol % Phosphate Buffered Saline: 50 vol % Visipaque contrast medium. The mixture was made from 6 ml of phosphate buffered saline (PBS) (GIBCO, Life Technologies, Inc. Rockville, Md.) and 6 ml. of VISIPAQUE™ 320 mg l/ml iodoxinole contrast medium (Amersham Health, Cork, Ireland). Based on the density of VISIPAQUE™ of approximately 1.3 g/ml and the density of PBS of approximately 1.0 g/ml the 50 vol %/50 vol % mixture of PBS and contrast medium was estimated to have a density of approximately 1.2 g/ml. The polymer from which the microparticles were made, 85 mol % PLA; 15 mol % PGA polymer had a density of approximately 1.3 g/ml. The 500-707 micron sized microparticles were placed into the mixture of PBS and contrast medium. Since the polymer from which these microparticles was made had a higher density than the mixture of PBS and contrast medium in which they had been placed. The microparticles, which were either suspended in the mixture or which were floating on top of the mixture, were determined to have a bulk density of less than 1.3 g/ml. This difference in the bulk density of the suspended or floating microparticles from the density of polymer from which these microparticles were made was attributed to the presence of void spaces in the microparticles.

The compression resistant microparticles that were either floating or suspended in the PBS and contrast medium mixture were drawn into a 20 ml polypropylene syringe (Tyco Healthcare/Kendall, Joliet, Ill.). The luer fitting of the syringe 20 ml syringe was placed onto one port of a four-way stopcock (Catalog Number 91045, Mallickrodt Critical Care, Glens Falls, N.Y.) and a 3 ml. polycarbonate syringe (Merit Medical Systems, South Jordan, Utah). was placed onto another port of the stopcock. The compression resistant microspheres were then transferred from the 20 ml syringe to the 3 ml syringe. The 20 ml syringe was then removed from the four-way valve port and was replaced by an angioplasty balloon inflation device (B. Braun Medical, Inc., Bethlehem, Pa.). An EXCELSIOR™ 1018 microcatheter (Boston Scientific, Fremont, Calif.) was placed on the last remaining port of the four-way valve. The EXCELSIOR™ 1018 microcatheter had a tapered section made of a rigid thermoplastic material located between the luer fitting on its proximal end and the beginning of the flexible section of the catheter. The tapered section of the catheter reduced from an inner diameter of approximately 4000 microns at the luer fitting to 480 microns where the flexible section of the catheter began. The distal end of the microcatheter was placed into a clean 150 ml glass beaker. The compression resistant microspheres were transferred from the 3 ml syringe into the microcatheter. The angioplasty balloon inflation device was then used to force 20 ml of water through the microcatheter. As the water and microspheres were passed through the microcatheter, a back-pressure of 18 atm registered on the pressure gauge which was mounted on the angioplasty balloon inflation device. The pressure on the angioplasty balloon inflation device did not bleed off, indicating that the microcatheter had become obstructed. No microspheres were observed being ejected from the distal end of the microcatheter into the glass beaker. The test described in Example 18 were performed at room temperature (approximately 21° C.).

### Example 20

### Compression Resistance Testing of Compressive and Compression Resistant Microspheres Using a Catheter with a 330 Micron Inner Diameter

CONTOUR SE™, compressible, polyvinyl alcohol (PVA) microspheres of 300-500 micron diameter were obtained (Catalog Number 76-122, Boston Scientific Corporation, Watertown, Mass.). Approximately 1 ml of these microspheres were suspended in a mixture of 50 vol % Phosphate Buffered Saline: 50 vol % VISIPAQUE™ contrast medium. The mixture was made from 6 ml of phosphate buffered saline (PBS) (GIBCO, Life Technologies, Inc. Rockville, Md.) and 6 ml of VISIPAQUE™ 320 mg l/ml iodoxinole contrast medium (Amersham Health, Cork, Ireland). The suspension of compressible microspheres was then loaded into a 20 ml polypropylene syringe (Tyco Healthcare/Kendall, Joliet, Ill.). The 20 ml syringe was placed onto one port of a four-way stopcock (Catalog Number 91045, Mallickrodt Critical Care, Glens Falls, N.Y.) and a 3 ml polycarbonate syringe (Merit Medical Systems Inc. South Jordan, Utah). was placed onto another port of the stopcock. Compressible microspheres were then transferred from the 20 ml syringe to the 3 ml syringe. The 20 ml syringe was then removed from the port of the four-way valve and was replaced by an angioplasty balloon inflation device (B. Braun Medical, Inc., Bethlehem, Pa.). An ELITE SPINNAKER® 1.8 F-S flow directed catheter with HYDROLENE® (Boston Scientific, Fremont, Calif.) was placed on the last remaining port of the four-way valve. The ELITE SPINNAKER® 1.8 F-S flow directed catheter with HYDROLENE® had a tapered section made of a rigid thermoplastic material located between the luer fitting on its proximal end and the beginning of the flexible section of the catheter. The tapered section of the catheter reduced from an inner diameter of approximately 4000 microns at the luer fitting to 330 microns where the flexible section of the catheter began. The distal end of the catheter was placed into a clean 150 ml glass beaker. The compressible microspheres were transferred from the 3 ml syringe into the microcatheter. The angioplasty balloon inflation device was then used to force approximately 20 ml of water through the microcatheter. As the water, microspheres, PBS, and contrast medium were passed through the microcatheter, less than 1 atm pressure registered on the pressure gauge which was mounted on the angioplasty balloon inflation device. This pressure gauge had a range of 0-30 atm. Microspheres were observed being ejected from the distal end of the microcatheter into the glass beaker, and it appeared that all of the microspheres had passed through the microcatheter after the approximately 20 ml of water had passed through the microcatheter.

The same procedure was then followed using compression resistant bioabsorbable microspheres of the present invention. Compression resistant, bioabsorbable microspheres of 85 mol % PLA: 15 mol % PGA were prepared using a polymer with an inherent viscosity of 0.65 dl/gm in chloroform at 30° C. They were sized dry using two sieving runs. In the first sieving run two standard testing sieves, a No.35 sieve and a No. 50 sieve conforming to ASTM standard specification E 11 were used. The two sieves were stacked together with the No. 35 sieve having approximately 500 micron sized openings on top and the No. 50 sieve having approximately 300 micron sized openings on the bottom. The microspheres were placed onto the No. 35 sieve and then both sieves, while still stacked, were agitated to encourage the microspheres smaller than 500 microns in diameter to migrate through the No. 35 sieve. Microspheres with diameters smaller than 500 microns but greater than approximately 300 microns accumulated on the surface of the screen of the sieve on the bottom of the stack, which was a No. 50 sieve with approximately 300 micron sized openings. The microspheres that had accumulated on the surface of the No. 50 sieve were then collected for the second sieving run. Through this process of sieving, these microspheres were determined to be in the approximately 300-500 micron size range. In the second sieving run, these approximately 300-500 micron microspheres were placed onto a No. 40 sieve with approximately 425 micron sized openings that conformed to ASTM standard specification El 1. The No. 40 sieve and microspheres were agitated to encourage the microspheres smaller than 425 microns to migrate through the No. 40 sieve. Microspheres with diameters greater than 425 microns accumulated on the surface of the screen. The microspheres of approximately 425-500 micron diameter that had accumulated on the surface of the No. 40 sieve were then collected for the in-vitro compression resistance test.

Approximately 0.1 gm of the 425-500 micron diameter compression resistant microspheres were put into approximately 12 ml of a mixture of 50 vol % Phosphate Buffered Saline: 50 vol % VISIPAQUE™ contrast medium. The mixture was made from 6 ml of phosphate buffered saline (PBS) (GIBCO, Life Technologies, Inc. Rockville, Md.) and 6 ml of VISIPAQUE™ 320 mg l/ml iodoxinole contrast medium (Amersham Health, Cork, Ireland). Based on the density of VISIPAQUE™ of approximately 1.3 g/ml and the density of PBS of approximately 1.0 g/ml the 50 vol %/50 vol % mixture of PBS and contrast medium was estimated to have a density of approximately 1.2 g/ml. The polymer from which the microspheres were made, 85 mol % PLA; 15 mol % PGA polymer had a density of approximately 1.3 g/ml. The approximately 425-500 micron diameter compression resistant microspheres were placed into the mixture of PBS and contrast medium. Since the polymer from which these microspheres were made had a higher density than the mixture of PBS and contrast medium in which they had been placed, microspheres which were either suspended in the mixture or which were floating on top of the mixture were determined to have a bulk density of less than 1.3 g/ml. This difference in the bulk density of the suspended or floating microspheres from the density of polymer from which these microspheres were made was attributed to the presence of void spaces in the microspheres.

The compression resistant microspheres which were either floating or suspended in the PBS and contrast medium mixture were drawn into a 20 ml polypropylene syringe (Tyco Healthcare/Kendall, Joliet, Ill.). The luer fitting of the syringe 20 ml. syringe was placed onto one port of a four-way stopcock (Catalog Number 91045, Mallickrodt Critical Care, Glens Falls, N.Y.) and a 3 ml polycarbonate syringe (Merit Medical Systems Inc., South Jordan, Utah). was placed onto another port of the stopcock. The compression resistant microspheres were then transferred from the 20 ml syringe to the 3 ml syringe. The 20 ml syringe was then removed from the four-way valve port and was replaced by an angioplasty balloon inflation device (B. Braun Medical, Inc., Bethlehem, Pa.). An ELITE SPINNAKER® 1.8 F-S flow directed catheter with HYDROLENE® (Boston Scientific, Fremont, Calif.) was placed on the last remaining port of the four-way valve. The ELITE SPINNAKER® 1.8 F-S catheter had a tapered section made of a rigid thermoplastic material located between the luer fitting on its proximal end and the beginning of the flexible section of the catheter. The tapered section of the catheter reduced from an inner diameter of approximately 4000 microns at the luer opening to 330 microns at the beginning of the flexible section of the catheter. The distal end of the catheter was placed into a clean 150 ml glass beaker. The compression resistant microspheres were transferred from the 3 ml syringe into the catheter. The angioplasty balloon inflation device was then used to force water through the catheter. As the water and microspheres were attempted to be passed through the microcatheter, a pressure of 20 atm registered on the pressure gauge which was mounted on the angioplasty balloon inflation device. The pressure on the angioplasty balloon inflation device did not bleed off, indicating that the microcatheter had become obstructed. Essentially no microspheres were observed being ejected from the distal end of the microcatheter into the glass beaker. The tests described in Example 19 were performed at room temperature (approximately 21° C.).

### Example 21

### Compression Resistantance Testing of Compressive and Compression Resistant Microspheres Using a Mesh Screen with 420 Micron Openings

CONTOUR SE™, compressible, polyvinyl alcohol (PVA) microspheres of 500-700 micron diameter were obtained (Catalog Number 76-130, Boston Scientific Corporation, Watertown, Mass.). Approximately 1 ml of these microspheres were suspended in a mixture of 50 vol % Phosphate Buffered Saline: 50 vol % VISIPAQUE contrast medium. The mixture was made from 6 ml of phosphate buffered saline (PBS) (GIBCO, Life Technologies, Inc. Rockville, Md.) and 6 ml of VISIPAQUE™ 320 mg l/ml iodoxinole contrast medium (Amersham Health, Cork, Ireland). Approximately 12 ml of the suspension of compressible microspheres and contrast medium with PBS was then loaded into a 20 ml polypropylene syringe (Tyco Healthcare/Kendall, Joliet, Ill.). The 20 ml syringe was placed onto a 13 mm diameter, stainless steel syringe filter (Catalog Number A-02928-10, Cole Parmer Instrument Co., Vernon Hills, Ill.) which contained a screen of 40 mesh stainless steel screen with approximately 420 micron openings (Catalog Number S-0770, Sigma Chemical Co., St. Louis, Mo.), which had been cut to fit into the syringe filter. The plunger of the syringe was then depressed at a rate so that the 12 ml of compressible microspheres and contrast medium with PBS was forced through the 40 mesh screen with 420 micron openings in approximately 5 seconds. The effluent from the distal end of the syringe filter was collected and was found to contain many compressible microspheres. The syringe filter was then opened and a small quantity of compressible microspheres were also found in and around the stainless steel mesh screen.

The same procedure was then followed using compression resistant bioabsorbable microspheres of the present invention. Compression resistant, bioabsorbable microspheres of 85 mol % PLA: 15 mol % PGA were prepared using a polymer with an inherent viscosity of 0.65 dl/gm in chloroform at 30° C. They were sized dry using two sieving runs. In the first sieving run two standard testing sieves, a No. 35 sieve and a No. 50 sieve conforming to ASTM standard specification E 11 were used. The two sieves were stacked together with the No. 35 sieve having approximately 500 micron sized openings on top and the No. 50 sieve having approximately 300 micron sized openings on the bottom. The microspheres were placed onto the No. 35 sieve and then both sieves, while still stacked, were agitated to encourage the microspheres smaller than approximately 500 microns to migrate through the No. 35 sieve. Microspheres smaller than approximately 500 microns but greater than approximately 300 microns in diameter accumulated on the surface of the screen of the sieve on the bottom of the stack, which was a No. 50 sieve with 300 micron sized openings. The microspheres that had accumulated on the surface of the No. 50 sieve were then collected for the second sieving run. Through this process of sieving, these microspheres were determined to be in the approximately 300-500 micron size range. In the second sieving run, these approximately 300-500 micron microspheres were placed onto a No. 40 sieve with approximately 425 micron sized openings that conformed to ASTM standard specification E 11. The No. 40 sieve and microspheres were agitated to encourage the microspheres smaller than 425 microns to migrate through the No. 40 sieve. Microspheres with diameters greater than approximately 425 microns accumulated on the surface of the screen. The microspheres of approximately 425-500 micron diameter that had accumulated on the surface of the No. 40 sieve were then collected for the in-vitro compression resistance test.

Approximately 0.1 gm of the 425-500 micron diameter compression resistant microspheres were put into approximately 20 ml of a mixture of 50 vol % phosphate buffered saline: 50 vol % VISIPAQUE™ contrast medium. The mixture was made from 10 ml of phosphate buffered saline (PBS) (GIBCO, Life Technologies, Inc. Rockville, Md.) and 10 ml of VISIPAQUE™ 320 mg l/ml iodoxinole contrast medium (Amersham Health, Cork, Ireland). Based on the density of VISIPAQUE™ of approximately 1.3 g/ml and the density of PBS of approximately 1.0 g/ml the 50 vol %/50 vol % mixture of PBS and contrast medium was estimated to have a density of approximately 1.2 g/ml. The polymer from which the microspheres were made, 85 mol % PLA; 15 mol % PGA polymer had a density of approximately 1.3 g/ml. The approximately 425-500 micron diameter compression resistant microspheres were placed into the mixture of PBS and contrast medium. Since the polymer from which these microspheres were made had a higher density than the mixture of PBS and contrast medium in which they had been placed, microspheres which were either suspended in the mixture or which were floating on top of the mixture were determined to have a bulk density of less than 1.3 g/ml. This difference in the bulk density of the suspended or floating microspheres from the density of polymer from which these microspheres were made was attributed to the presence of void spaces in the microspheres.

Approximately 12 ml of a mixture of compression resistant microparticles which were either floating or suspended in the PBS and contrast medium mixture and PBS with contrast medium were drawn into a 20 ml polypropylene syringe (Tyco Healthcare/Kendall, Joliet, Ill.). The luer filting of the 20 ml disposable syringe was placed onto a 13 mm diameter, stainless steel syringe filter (Catalog Number A-02928-10, Cole Parmer Instrument Co., Vernon Hills, Ill.) which contained a screen of 40 mesh stainless steel screen with 420 micron sized openings (Catalog Number S-0770, Sigma Chemical Co., St. Louis, Mo.). that had been cut to fit into the syringe filter. The plunger of the syringe was then depressed at a rate so that the 12 ml of compression resistant microspheres and contrast medium with PBS was forced through the 40 mesh screen with 420 micron openings in approximately 5 seconds. The effluent from the distal end of the syringe filter was collected and was found to contain essentially no compression-resistant microspheres. The syringe filter was then opened and many compression resistant microspheres were found in and around the stainless steel mesh screen. The testing described in Example 20 was performed at room temperature (approximately 21° C.).

### Example 22

### Microsphere Fabrication

Microspheres as used in testing for compression resistance as described in Examples 18-20 were prepared generally according to the following process:

1) 1.2 gm of 85/15 poly (DL-lactide-co-glycolide) polymer (Absorbable Polymers International, Pelham, Ala.) was put into a 10 ml glass beaker.

2) 7.5 ml of ethyl acetate (Fisher Scientific, Fair Lawn, N.J.) was added to the glass beaker containing the polymer.

3) The beaker containing the ethyl acetate and polymer was covered with PARAFILM® M (American National Can, Neenah, Wis.) and left overnight (approximately 12 hours) at room temperature (approximately 21° C.).

4) 10.5 ml of ethyl acetate was mixed into 150 ml of DI water in a 1000 ml glass beaker which contained a 1.5 inch long TEFLON® coated magnetic stir bar (Cole Parmer Instrument Co, Vemon Hills, Ill.). The beaker with DI water and ethyl acetate was put onto a magnetic stir plate (Model 546725, Bamstead/Thermolyne, Dubuque, Iowa) with a speed setting of about “3”. The ethyl acetate and DI water was allowed to mix for 30 minutes.

5) The polymer and ethyl acetate solution was then poured from the 10 ml beaker into the mixture of ethyl acetate and DI water in the 1000 ml beaker. This took approximately 10-15 seconds and the magnetic stirrer was continuing to stir during this time. Polymer microspheres formed at this time.

6) Approximately 15 seconds after the polymer and ethyl acetate solution had been completely poured into the mixture of ethyl acetate and DI water, 650 ml of DI water was then added to the 1000 ml beaker and the magnetic stirrer speed was increased to a setting of about “7”.

7) This mixture was allowed to stir for 12 hours at room temperature (approximately 21° C.), during which time the microspheres were allowed to harden.

8) The microspheres were then sieved using an ASTM E-11 No. 450 sieve with 32 micron openings (U.S. Standard Sieve Series, Dual Mfg. Co., Chicago, Ill.).

9) The microspheres were washed in the sieve with copious amounts of DI water.

10) The microspheres were then transferred to a screw cap plastic vial.

11) The microspheres were immediately frozen at −80° C.

12) The microspheres were lyophilized overnight (approximately 12 hours) and then stored refrigerated (approximately 3° C.).

While particular embodiments of the present invention have been illustrated and described herein, the present invention should not be limited to such illustrations and descriptions. It should be apparent that changes and modifications may be incorporated and embodied as part of the present invention within the scope of the following claims.

1. A microparticle comprising:
a homopolymer of at least a poly(a-hydroxy ester) or a copolymer of at least a poly(a-hydroxy ester) having a trimethylene carbonate moiety; the microparticle adapted for catheter delivery.

2. A microparticle according to claim 1, wherein said microparticle is an embolic agent.

3. A microparticle according to claim 1, wherein said microparticle is substantially neutrally buoyant relative to a target bodily fluid for a time necessary to deliver a bolus of particles.

4. A microparticle according to claim 1, having at least one bioactive agent.

5. A microparticle according to claim 1, having at least one additive.

6. A microparticle according to claim 1, further comprising at least one coating.

7. A microparticle according to claim 1, having a number of distributed voids.

8. A microparticle according to claim 1, having a convoluted surface.

9. A catheter deliverable microparticle comprising: at least one bioresorbable base polymer and a void volume, wherein the microparticle is compression resistant.

10. A microparticle according to claim 9, wherein said microparticle is an embolic agent.

11. A microparticle according to claim 9, wherein said microparticle is substantially neutrally buoyant relative to a target bodily fluid for a time necessary to deliver a bolus of particles.

12. A microparticle according to claim 9, having at least one bioactive agent.

13. A microparticle according to claim 9, having at least one additive.

14. A microparticle according to claim 9, further comprising at least one coating.

15. A microparticle according to claim 9, having a number of distributed voids.

16. A microparticle according to claim 9, having a convoluted surface.

17. A bolus of embolic microparticles, comprising: a plurality of catheter deliverable microparticles, said catheter deliverable microparticles being compression resistant and having at least one bioresorbable base polymer and having a void volume.

18. A bolus of embolic microparticles according to claim 17, wherein a number of said catheter deliverable microparticles have a density which is between 0.9 g/cc and 1.4 g/cc.

19. A bolus of embolic microparticles according to claim 17, wherein a number of said catheter deliverable microparticles have a specific gravity of 0.6 to 1.4 relative to a target bodily fluid.

20. A bolus of embolic microparticles according to claim 17, wherein a therapeutically effective number of said catheter deliverable microparticles are neutrally buoyant relative to a target bodily fluid.

21. A bolus of embolic microparticles according to claim 17, wherein a number of said catheter deliverable microparticles have at least one bioactive agent.

22. A bolus of embolic microparticles according to claim 17, wherein a number of said catheter deliverable microparticles have an external diameter and are resistant to a deformation of said external diameter of greater than 10%.

23. A catheter deliverable microparticle comprising:
at least one bioresorbable base polymer,
a second material which is different from said at least one bioresorbable base polymer,
a void volume in which said second material is present, and
wherein said microparticle is compression resistant.

24. A method of making microparticles comprising
providing an organic solvent;
mixing the organic solvent and a solvated polymer in an aqueous solution;
maintaining the organic solvent in the aqueous solution in a quantity and under conditions that maintain the organic solvent below an organic solvent saturation point of the aqueous solution; and
creating microparticles in the aqueous solution while maintaining the solution below the saturation point.

25. The method of claim 24 that further comprises providing as the organic solvent ethyl acetate.

26. The method of claim 24 that further comprises providing as the organic solvent methylene chloride.

27. A method of testing a microparticle for compression resistance comprising
providing a sample of microparticles suspended in a solution, the microparticles having a given nominal cross-sectional dimension of x;
providing a test apparatus that includes at least one constriction with an effective opening size with a dimension less than x;
passing the sample of microparticles and solution through the at least one constriction in the test apparatus under pressure;
sampling the solution downstream of the at least one constriction to determine if the microparticles passed through the constriction without permanent damage to the microparticles;
wherein particles of dimension x that do not pass through the constriction are deemed compression-resistant.
